(19) United States
(12) Patent Application Publication
(10) Pub. No.: US 2026/0139304 A1
(43) Pub. Date: May 21, 2026

(54) MODIFIED NUCLEOTIDES TO REDUCE PRIMER DIMER FORMATION IN MULTIPLEX PCR

(71) Applicant: BillionToOne, Inc., Menlo Park, CA (US)

(72) Inventors: Devon Brian Chandler BROWN, Campbell, CA (US); Patrick YE, Palo Alto, CA (US); David TSAO, San Carlos, CA (US); Oguzhan ATAY, Burlingame, CA (US)

(21) Appl. No.: 18/954,463

(22) Filed: Nov. 20, 2024

(51) Int. Cl. C12Q 1/6853; C12Q 1/686

(52) U.S. Cl. CPC C12Q 1/6853; C12Q 1/686

(57) ABSTRACT

Multiplex PCR amplification may be improved by using primers with exonuclease-resistant bonds or nucleotides to reduce the formation of primer dimers. Multiplex PCR-based assays may be improved by using a load-balanced set of primers with different proportions of primers in the set to provide balanced amplicon read depth.

### BACKGROUND

### Sequence Listing

The instant application contains a Sequence Listing which has been submitted electronically in XML format and is hereby incorporated by reference in its entirety. Said XML copy, created on Jan. 29, 2025, is named 38227_59424_US.xml and is 5,141 bytes in size.

### TECHNICAL FIELD

The subject matter described herein relates generally to multiplex PCR amplification, and in particular to optimization of multiplex PCR amplification reactions.

### BACKGROUND INFORMATION

Multiplex polymerase chain reaction (PCR) enables simultaneous amplification of multiple genomic targets in a single reaction. This can be particularly useful when there is limited volume of the input sample; splitting a sample into multiple singleplex PCRs would reduce the amount of input sample per reaction, worsening the performance of the assay. Furthermore, performing multiple reactions for a single sample can become logistically impractical in a clinical laboratory setting.

One technical challenge with performing multiplex PCR is the rate of primer dimer formation. Primer dimers are undesired amplification products that form when two primer oligonucleotides anneal to each other and amplify, instead of amplifying the desired genomic targets. In a poorly performing multiplex PCR, the products can be composed of more than 90% primer dimers. This is particularly challenging as the number of targets in the multiplex PCR increases; the number of potential primer dimers is proportional to the square of the number of primers.

A common situation that arises in multiplex PCR is when several bases worth of sequence at the 3′ ends of two primer oligonucleotides are perfectly complementary. During PCR, the 3′ ends anneal, and the polymerase extends to double strand both the primers. Methods to minimize this primer dimer situation include in silico detection of 3′ sequence complementarity. This can be computationally complex because one has to check for complementarity for each combination of two primers (including one primer against itself) for a varying number of bases at the 3′ end of each primer. The complexity is a function of the number of primers squared.

An alternative situation is when the 3′ ends of two primer oligonucleotides are not perfectly complementary. This can still lead to primer dimer formation if a polymerase with 3′ to 5′ exonuclease activity, as found in proofreading polymerases, chews back the non-complementary sequence. Removal of mismatching nucleotides now results in perfectly complementary sequence on the 3′ ends, allowing for primer dimer amplification. This further increases the computational intensity of checking for complementarity in silico, as the computation would now additionally require removing a varying number of bases from the 3′ ends of each primer. Thus, in silico testing of large numbers (e.g., hundreds) of primers is infeasible.

Another technical challenge with performing multiplex PCR is variance in amplicon read depth across primer pairs arising from differences in amplification efficiency and second-order effects between different primer pairs. Furthermore, depending on the difference in abundance of a target sequence between a positive and negative call, different read depths may be desirable for different amplicons in a multiplexed assay (for example, simply detecting the presence of a target sequence may be sufficient for one test in a multiplexed assay while another may require differentiating small differences in target counts, with the latter requiring a significantly greater read depth than the former to make a determination with a desired degree of statistical certainty). There is an on-going need for improved multiplex PCR techniques.

### SUMMARY

Exonuclease-resistant primers multiplex may be used in multiplex PCR amplification to reduce primer dimer formation. In one embodiment, an exonuclease-resistant primer includes one or more exonuclease-resistant nucleotides. Additionally or alternatively, the exonuclease-resistant primer may include one or more exonuclease-resistant internucleotide bonds. The efficiency of the multiplex PCR amplification may also be improved by using load balancing of primer concentrations to obtain desired amplicon read depths for each of a set of amplicons. For example, the load balancing may be used to provide approximately the same read depth for each read or provide sufficient read depth for each amplicon in an assay in view of expected signal strength.

In some embodiments, a method of amplifying at least 50 or at least 100 target loci in a nucleic acid sample comprises performing multiplex PCR amplification on the nucleic acid sample using a pool of primers comprising a set of primer pairs collectively comprising at least one primer pair for each of the at least 50 target loci, wherein each of a subset of primers in the pool of primers comprises at least one exonuclease-resistant internucleotide bond. Each of the plurality of primers may comprise an exonuclease-resistant internucleotide bond between: (a) a nucleotide at the 3′-terminal position (0 position) and the nucleotide immediately upstream of the 0 position (−1 position); (b) a nucleotide at the −1 position and the nucleotide immediately upstream of the −1 position (−2 position); (c) a nucleotide at the −2 position and the nucleotide immediately upstream of the −2 position (−3 position); or (d) a nucleotide at the −3 position and the nucleotide immediately upstream of the −3 position (−4 position).

The method may further comprise treating the pool of primers with an enzyme having a 3′ to 5′ exonuclease activity prior to performing the multiplex PCR amplification. Following enzymatic treatment, less than 1% of the primers remaining in the pool of primers do not have at least one exonuclease-resistant internucleotide bond. The exonuclease-resistant internucleotide bond may be a phosphorothioate bond.

The plurality of primers may comprise at least 90% of the primers in the pool of primers. The amount of primer dimer formation in the multiplex PCR amplification may be less than 70% of the amount of primer dimer formation that would occur in a corresponding multiplex PCR amplification where each of the primers in the pool of primers has only phosphodiester internucleotide bonds. The percent of primer dimers in the products of the multiplex PCR amplification may be less than 50%. Each of a second subset of the pool of primers may have three or more phosphorothioate bonds, with each phosphorothioate bond being an Rp isomer or an Sp isomer, such that statistically a predetermined percentage of the second subset of the pool of primers have at least one Sp isomer phosphorothioate bond. The subset of primers that have at least one exonuclease-resistant internucleotide bond includes those of the second subset of primers that have at least one Sp isomer phosphorothioate bond.

At least some of the subset of primers in the pool of primers may comprise an exonuclease-resistant nucleotide. The exonuclease-resistant nucleotide may be at: (a) the 3′-terminal position (0 position); (b) the nucleotide immediately upstream of the 0 position (−1 position); (c) the nucleotide immediately upstream of the −1 position (−2 position); or (d) the nucleotide immediately upstream of the −2 position (−3 position). The exonuclease-resistant nucleotide may have a sugar moiety with a bridge connecting the 2′ oxygen and 4′ carbon.

The pool of primers may be a load-balanced set of primer pairs, wherein concentrations of the primer pairs was determined by a process comprising: (i) performing a multiplex PCR amplification on a first balancing nucleic acid sample using an initial pool of primers comprising a set of initial primer pairs collectively comprising at least one primer pair for each of the plurality of target loci, wherein each initial primer pair is present in the initial pool of primers at a concentration according to a first primer pair concentration profile, thereby generating amplicons corresponding to the different primer pairs; (ii) determining sequence read depths of amplicons from the multiplex PCR amplification corresponding to the different primer pairs to generate a first amplification profile; (iii) generating an updated primer pair concentration profile based on the first amplification profile, wherein concentrations in the updated primer pair concentration profile are selected based on the first amplification profile to adjust the sequence read depths to be closer to corresponding target sequence read depths for the primer pairs; and (iv) determining the concentrations of the primer pairs for the load-balanced set of primer pairs based on the updated primer pair concentration profile.

In other embodiments, a method of amplifying a plurality of target loci in a nucleic acid sample comprises: performing multiplex PCR amplification on the nucleic acid sample using a pool of primers comprising a set of primer pairs collectively comprising at least one primer pair for each of the plurality of target loci, wherein each of a plurality of primers in the pool of primers comprises at least one exonuclease-resistant nucleotide, wherein: (i) the plurality of target loci comprises at least 50 target loci or at least 100 target loci; or (ii) the at least one exonuclease-resistant nucleotide comprises a plurality of exonuclease-resistant nucleotides. Each of the plurality of primers may comprise an exonuclease-resistant nucleotide at: (a) the 3′-terminal position (0 position); (b) the nucleotide immediately upstream of the 0 position (−1 position); (c) the nucleotide immediately upstream of the −1 position (−2 position); or (d) the nucleotide immediately upstream of the −2 position (−3 position).

The method may further comprise treating the pool of primers with an enzyme having a 3′ to 5′ exonuclease activity prior to performing the multiplex PCR amplification. Following enzymatic treatment, less than 1% of the primers remaining in the pool of primers do not have at least one exonuclease-resistant nucleotide. The exonuclease-resistant nucleotide may have a sugar moiety with a bridge connecting the 2′ oxygen and 4′ carbon. The plurality of primers may comprise at least 90% of the primers in the pool of primers and the amount of primer dimer formation in the multiplex PCR amplification may be less than 70% of the amount of primer dimer formation in a corresponding multiplex PCR amplification where each of the primers in the pool of primers has only natural nucleotides. The percent of primer dimers in the products of the multiplex PCR amplification may be less than 50%.

At least some of the plurality of primers in the pool of primers may comprise an exonuclease-resistant internucleotide bond. The exonuclease-resistant bond may be between: (a) a nucleotide at the 3′-terminal position (0 position) and the nucleotide immediately upstream of the 0 position (−1 position); (b) a nucleotide at the −1 position and the nucleotide immediately upstream of the −1 position (−2 position); (c) a nucleotide at the −2 position and the nucleotide immediately upstream of the −2 position (−3 position); or (d) a nucleotide at the −3 position and the nucleotide immediately upstream of the −3 position (−4 position). The exonuclease-resistant internucleotide bond may be a phosphorothioate bond. Each of a subset of the pool of primers may have three or more phosphorothioate bonds, each phosphorothioate bond being an Rp isomer or an Sp isomer, such that statistically a predetermined percentage of the second subset of the pool of primers have at least one Sp isomer phosphorothioate bond. The subset of primers that have at least one exonuclease-resistant internucleotide bond includes those of the second subset of primers that have at least one Sp isomer phosphorothioate bond.

The pool of primers may be a load-balanced set of primer pairs, wherein concentrations of the primer pairs in the load-balanced set of primers was determined by a process comprising: (i) performing a multiplex PCR amplification on a first balancing nucleic acid sample using an initial pool of primers comprising a set of initial primer pairs collectively comprising at least one primer pair for each of the plurality of target loci, wherein each initial primer pair is present in the initial pool of primers at a concentration according to a first primer pair concentration profile, thereby generating amplicons corresponding to the different primer pairs; (ii) determining sequence read depths of amplicons from the multiplex PCR amplification corresponding to the different primer pairs to generate a first amplification profile; (iii) generating an updated primer pair concentration profile based on the first amplification profile, wherein concentrations in the updated primer pair concentration profile are selected based on the first amplification profile to adjust the sequence read depths to be closer to corresponding target sequence read depths for the primer pairs; and (iv) determining the concentrations of the primer pairs for the load-balanced set of primer pairs based on the updated primer pair concentration profile. At least some of the primers may include at least one exonuclease-resistant nucleotide and at least one exonuclease-resistant internucleotide bond, thereby balancing impacts on primer annealing temperature and sequence read depths of amplicons caused by the at least one exonuclease-resistant nucleotide and the at least one exonuclease-resistant internucleotide bond. The at least one exonuclease-resistant nucleotide may have a sugar moiety with a bridge connecting the 2′ oxygen and 4′ carbon and the at least one exonuclease-resistant internucleotide bond may be a phosphorothioate bond.

### BRIEF DESCRIPTION OF THE DRAWINGS

FIG. 1 illustrates two primer oligonucleotides (Primer 1: SEQ ID NO: 1, Primer 2: SEQ ID NO: 2) with perfectly complementary sequences on their 3′ ends. Arrows indicate direction of polymerase extension after annealing.

FIG. 2A illustrates two primer oligonucleotides (Primer 1: SEQ ID NO: 3; Primer 2: SEQ ID NO: 2) with one mismatching base at the 3′ end of Primer 1. FIG. 2B illustrates the two primers (Primer 1: SEQ ID NO: 4; Primer 2: SEQ ID NO: 2) of FIG. 2A following processing with a polymerase having 3′ to 5′ exonuclease activity that detects the mismatch and chews back the mismatched nucleotide. This results in perfectly complementary sequence on the 3′ ends and allows for primer dimer amplification. Arrows indicate the direction of polymerase extension after annealing.

FIG. 3 shows chemical structures of phosphodiester and phosphorothioate bonds.

FIG. 4 shows chemical structures illustrating two isomers for phosphorothioate bonds, with one isomer being resistant to exonuclease activity and the other being not resistant, according to one embodiment.

FIG. 5 shows the percentage of primer dimers as a function of the number of phosphorothioate bonds at the 3′ end of primers and exonuclease pre-treatment for a multiplex PCR using 16 primers, according to one embodiment.

FIG. 6A is a plot of yield for a multiplex PCR reaction with more than 500 primer pairs that includes the same primer sequences but with varying numbers of consecutive phosphorothioate bonds at the 3′ end of each primer. FIG. 6B is a plot of on-target rate for the multiplex PCR reaction of FIG. 6A.

FIG. 7 illustrates gel electrophoresis results for 16 primer pair multiplex PCR products with primers with no modifications (vanilla), four consecutive phosphorothioate bonds at the 3′ end of each primer (PTO), and having a locked nucleic acid at the penultimate 3′ end of each primer (LNA). The on-target bands (˜130 bp) for the LNA primer conditions are significantly more prominent than the on-target bands for the vanilla and PTO primer conditions. The off-target bands (<100 bp) for the LNA primer conditions are fewer and more distinct when compared to the off-target bands for the vanilla and PTO primer conditions.

FIG. 8 illustrates on-target rate as measured by next-generation sequencing of 16 primer pair multiplex PCR with primers with no modifications (vanilla), four consecutive phosphorothioate bonds at the 3′ end of each primer (PTO), and having a locked nucleic acid at the penultimate 3′ end of each primer (LNA).

FIG. 9A shows the relationship between primer concentration and read depth for two primer pairs, pa18 and pa3, plotted using linear axes. FIG. 9B shows the relationship between primer concentration and read depth for pa18 and pa3 plotted using log-log axes.

FIG. 10 shows the relationship between primer concentration and read depth for 19 primer pairs plotted using log-log axes.

FIG. 11 shows the modelled relationship between primer concentration and amplification efficiency for each of a set of primers.

FIG. 12 shows the read depth obtained for each other primer pair in the set as the concentration of primer pair pa18 is varied, illustrating that there are not significant second order effects where the concentration of pa18 significantly impacts the obtained rad depth for other primers.

FIG. 13 shows the impact on read depth for each primer pair as the concentration of each other primer is varied, illustrating that there are not significant second order effects between primer pairs in the set.

FIG. 14 is a plot of the measured ratio of a reference to a corresponding spike-in against PCR efficiency for each primer pair in the set illustrating that there is not a consistent relationship between amplification efficiency and bias.

FIG. 15 illustrates that the modification of primers with LNAs can improve the amplification efficiency or evenness, according to one embodiment.

FIG. 16 illustrates that the modification of primers with LNAs can improve capture performance, according to one embodiment.

### DETAILED DESCRIPTION

The figures and the following description describe certain embodiments by way of illustration only. One skilled in the art will readily recognize from the following description that alternative embodiments of the structures and methods may be employed without departing from the principles described. Wherever practicable, similar or like reference numbers are used in the figures to indicate similar or like functionality. Where elements share a common numeral followed by a different letter, this indicates the elements are similar or identical. A reference to the numeral alone generally refers to any one or any combination of such elements, unless the context indicates otherwise.

### Overview

The present disclosure provides a method of performing multiplex PCR amplification on a nucleic acid sample where at least a subset of the primers used includes an exonuclease-resistant nucleotide or internucleotide bond. An example of an exonuclease-resistant internucleotide bond is a phosphorothioate bond. An example of an exonuclease-resistant nucleotide is a nucleotide having a sugar moiety with a bridge connecting the 2′ oxygen and 4′ carbon, also referred to as a locked nucleic acid (LNA) nucleotide. However, other types of bond or nucleotide that are resistant to exonuclease digestion may be used.

As discussed above, a common situation that may contribute to primer dimer formation in multiplex PCR reactions is where the 3′ ends of two primers are not initially perfectly complementary, but removal of mismatching nucleotides by a polymerase with 3′ to 5′ exonuclease activity leads to perfectly complementary 3′ ends and primer dimer formation and amplification. Reducing the extent to which primers can be chewed back at their 3′ ends can limit the amount of primer dimers forming because of this situation, improving multiplex PCR efficiency and reducing imbalances in read depths of amplicons associated with susceptible primers.

Additionally or alternatively, the multiplexed PCR amplification may be improved by balancing the amount of each primer pair in view of a corresponding target read depth for the primer pair. Primer pairs amplify target sequences in template DNA during a multiplex polymerase chain reaction (PCR), but they may do so with different efficiencies. Because PCR involves exponential amplification, even a small difference in amplification efficiency can result in very large differences in product formation. When next-generation sequencing (NGS) libraries are prepared from multiplex PCR, amplification variability across primer pairs can result in some targets getting orders of magnitude more reads than others. The concentration of a primer pair in a PCR reaction also affects amplification efficiency, with more concentrated primers resulting in greater amplification up to a certain point. This means that altering the primer pair concentration is one way to modulate the proportion of reads that a given target gets during NGS.

In various embodiments, to account and correct for amplification variability, rebalancing experiments can be performed in which the concentration of primers are altered according to previous empirical data. Specifically, when targets receive higher than the desired output (e.g. number of reads) on a sequencing run, the corresponding primer pair for this target can be reduced in concentration to decrease the proportion of reads in the library. In one embodiment, primers are randomly assigned to and pooled into groups. The primer concentrations for one group of primers are varied while the concentrations of the other groups of primers remains constant. This is repeated for each group of primers so that each primer is tested at variable concentrations. These primer conditions can then be tested via multiplex PCR in an organized fashion on a rebalancing place (e.g., a 96-well plate). The primer mixes in the rebalancing plate are used in separate PCR reactions, the products of which are sequenced using NGS to create a titration curve relating primer concentration and target output. For each primer pair, a new concentration may be selected, or the concentration could remain unchanged if the target is already at the desired output.

The new chosen primer concentration may be selected using multiple approaches based on what is appropriate for the particular purpose of the PCR reaction. In one embodiment, a tested concentration for a primer pair that is closest to the target read depth on the titration curve is selected as the new concentration. In another embodiment, the new concentration is selected using linear interpolation on the titration curve to predict a concentration that will provide the target read depth. In other embodiments, other interpolation methods may be used to predict primer pair concentrations that will provide the corresponding target read depths.

This process can be iterated until a set of primer pair concentrations that provides the desired output for each primer pair (within a predefined acceptable error range) for each primer pair. Once the desired output is achieved, the sets of primers with the determined concentrations may be prepared for future use in assays. The desired output may be selected based on the assays for which the primer pairs will ultimately be used for. For example, an assay may define a target read depth for each of a set of amplicons, with the read depth depending on a degree of sensitivity/accuracy required for the assay. Thus, the primer rebalancing technique may be used to design custom primer pair mixtures for specific assays that can then be produced in bulk for future use. In some embodiments, variation on the precise balance of primers in a bulk-produced batch may be accounted for by using the same rebalancing technique (or a similar technique) to the one used to determine the desired concentrations for the primers. Because the bulk-produced batch typically starts close to the desired concentrations, the rebalancing may be performed relatively quickly with only a small number of (e.g., one or two) iterations.

### Example Methods of Reducing Primer Dimer Formation

In a multiplexed PCR process, a set of primers is prepared for amplifying a set of amplicons. In various embodiments, an exonuclease-resistant nucleotide or internucleotide bond is included in at least a subset of the primers (e.g., at the 3′ terminus of a primer). For example, a primer of the subset of primers can have an exonuclease-resistant internucleotide bond between the 3′ terminal nucleotide and the nucleotide directly preceding (i.e., 5′ of) the 3′ terminal nucleotide. Also encompassed are primers where the exonuclease-resistant internucleotide bond may be present between any two adjacent nucleotides of the five 3′ terminal nucleotides. Similarly, a primer of the subset of primers can have an exonuclease-resistant nucleotide at the 3′ terminal position, or any of the four 3′ terminal positions. In some embodiments, a primer of the subset of primers can have both an exonuclease-resistant internucleotide bond and an exonuclease-resistant nucleotide.

### Exonuclease-Resistant Internucleotide Bond

A set of target loci in a nucleic acid sample may be amplified by performing multiplex PCR amplification on the nucleic acid sample using a pool of primers. In one embodiment, the pool of primers includes a set of primer pairs with at least one primer pair for each of the target loci. At least some of the primers in the pool (i.e., a subset of the primers) have at least one exonuclease-resistant internucleotide bond. The set of target loci may include at least 50 (such as at least any of 50, 100, 200, 300, 400, 500, 600, 700, 800, 900, 1000, or more) target loci. The exonuclease-resistant internucleotide bond in a primer may be at the 3′ end of the primer. In this context, the 3′ end of a primer includes the five 3′ terminal nucleotides. Some or all of the subset of primers may have multiple (e.g., 2, 3, 4, 5, or more) exonuclease-resistant internucleotide bonds.

In some embodiments, a method of amplifying a plurality of target loci uses a pool of primers, a subset of which have an exonuclease-resistant internucleotide bond. Each primer of the subset of primers has an exonuclease-resistant internucleotide bond between (a) the nucleotide at the 3′-terminal position (referred to herein as the “0 position”) and the nucleotide immediately upstream of the 0 position (referred to herein as the “−1 position”); (b) the nucleotide at the −1 position and the nucleotide immediately upstream of the −1 position (referred to herein as the “−2 position”); (c) the nucleotide at the −2 position and the nucleotide immediately upstream of the −2 position (referred to herein as the “−3 position”); or (d) the nucleotide at the −3 position and the nucleotide immediately upstream of the −3 position (referred to herein as the “−4 position”). In other words, the primer may have an exonuclease-resistant internucleotide bond between the nucleotides at the 0 and −1 positions the −1 and −2 positions the −2 and −3 positions, or the −3 and −4 positions.

The exonuclease-resistant internucleotide bond may be a phosphorothioate bond. There are two isomers for phosphorothioate bonds (the Rp isomer and the Sp isomer), with the Sp isomer being significantly more (approximately 300 times mores) resistant to exonuclease activity. With current DNA synthesis technology, the phosphorothioate bond isomer that is added to the 3′ end of an oligonucleotide is random, resulting in only about 50% of phosphorothioate bonds in synthesized primers being exonuclease-resistant bonds. Therefore, about 50% of primers with one phosphorothioate bond will be susceptible to 3′ exonuclease digestion, about 25% of primers with two phosphorothioate bonds will be susceptible to 3′ exonuclease digestion, about 12.5% of primers with three phosphorothioate bonds will be susceptible to 3′ exonuclease digestion, and so on. Thus, the proportion of instances of a primer that are resistant versus susceptible to exonuclease digestion may be tailored by controlling the number of phosphorothioate bonds. Alternatively, processes exist for adding only Sp Isomer phosphorothioate bonds, which provide another mechanism for tailoring primers to have a specific number of phosphorothioate bonds.

In some embodiments, a primer of the subset of primers comprises 1, 2, 3, or 4 phosphorothioate internucleotide bonds. For example, a primer with two internucleotide phosphorothioate bonds may have phosphorotbioate bonds between the nucleotides at the 0 and −1 positions and the nucleotides at the −1 and −2 positions, a primer with three internucleotide internucleotide phosphorothioate bonds may have phosphorothioate bonds between the nucleotides at the 0 and −1 positions, the nucleotides at the −1 and −2 positions, and the nucleotides at the −2 and −3 positions, and a primer with four internucleotide phosphorothioate bonds may have phosphorothioate bonds between the nucleotides at the 0 and −1 positions, the nucleotides at the −1 and −2 positions, the nucleotides at the −2 and −3 positions, and the nucleotides at the −3 and −4 positions.

In some embodiments, the method also includes treating the pool of primers with an enzyme having a 3′ to 5′ exonuclease activity (e.g., a DNA polymerase I, a T7 DNA polymerase, a Klenow fragment, a vent DNA polymerase, a Pfu DNA polymerase, or any other suitable enzyme) prior to performing the multiplex PCR amplification. For example, the pool of primers can be treated with the enzyme having a 3′ to 5′ exonuclease activity under conditions that allow for at least 50% (such at least any of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) of primers in the pool of primers having no exonuclease-resistant internucleotide bonds to be digested. Following enzymatic treatment, the primers remaining from the pool of primers may include less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%) that have at least one exonuclease-resistant internucleotide bond. In some embodiments, the enzyme-treated primers are purified prior to use in the multiple PCR amplification. The enzyme-treated primers may be used in the multiple PCR amplification following inactivation (e.g., heat inactivation) of the enzyme.

In some embodiments, according to any of the methods of amplifying a plurality of target loci described herein employing a subset of primers comprising an exonuclease-resistant internucleotide bond, the subset of primers corresponds to at least 50% (such at least any of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) of the primers in the pool of primers.

In some embodiments, the method further includes determining the amount of primer dimer formation following the multiplex PCR amplification. The amount of primer dimer formation may be determined using next generation sequencing (NGS). For example, once the multiplex PCR amplification has been carried out, the resulting amplicons can be sequenced using NGS technology, and sequencing reads can be mapped back to the sequences of primers from the pool of primers. with a primer dimer sequence can be identified as one having full or partial alignment to two primer sequences. These reads can be counted or quantified to determine the extent of primer dimer formation. Alternatively, determining the amount of primer dimer formation is carried out by gel electrophoresis or high-resolution melting analysis (HRMA). The amount of primer dimer formation in the multiplex PCR amplification may be characterized by the percentage of sequence reads that correspond to primer dimers. Using the disclosed techniques, this percentage may be less than 50% (such as less than any of 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amount of primer dimer formation in the multiplex PCR amplification is characterized by a percentage of sequence reads corresponding to primer dimers of less than 70% (such as less than any of 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) of the amount of primer dimer formation in a corresponding multiplex PCR amplification where each of the primers in the pool of primers has only phosphodiester internucleotide bonds.

In some embodiments, once the amount of primer dimer formation has been determined following a multiplex PCR amplification for a subset of primers in the pool of primers, the method may further include generating a filtered set of primer pairs based on the set of primer pairs in the pool of primers that excludes, for each primer dimer found to be present above a predetermined threshold, at least one of the primer pairs having a primer in the corresponding primer dimer. For example, where primer pairs A-for/A-rev and B-for/B-rev are present in the pool of primers, and a primer dimer is determined to have formed above a predetermined threshold amount from A-for and B-rev following multiplex PCR amplification, either A-for/A-rev or B-for/B-rev can be excluded in the filtered set of primer pairs. The predetermined threshold can be a fraction of the total reads for a given sample (e.g., more than 1%, more than 0.1%, or more than 0.01%, etc.). The threshold may depend on the overall primer-dimer rates and the number of primer-dimer species. The contribution of particular primers to all of the primer dimers detected in the sequence read results may be calculated and used to determine which primers to remove. For example, whether A-for/A-rev or B-for/B-rev is removed in the example given above may be selected based on which primer contributes most of the overall creation of primer dimers, accounting for other primer dimers that each contributes to.

In some embodiments, the amplicons generated in the multiplex PCR amplification can be sequenced using NGS. The sequencing generates sequence read data for the amplicons. The sequencing may involve preprocessing the amplicons to adapt them for NGS sequencing. The sequence read data may be used to determine sequence read depths for the amplicons.

In some embodiments, according to any of the methods of amplifying a plurality of target loci described herein employing a subset of primers comprising an exonuclease-resistant internucleotide bond, the method further comprises using the multiplex PCR amplification in a downstream application (e.g., a diagnostic or other assay).

Using some or all of these techniques, a multiplex PCR method may amplify at least 50 target loci in a nucleic acid sample using a pool of primers. In some embodiment, the pool of primers includes a set of primer pairs collectively comprising at least one primer pair for each of the target loci. Each primer of a subset of the primers in the pool includes at least one exonuclease-resistant internucleotide bond (e.g., a phosphorothioate internucleotide bond) at the 3′ end of the primer, such as at one or more of the five 3′ terminal nucleotides. The subset of primers may include at least 50% of the primers in the pool of primers. In some embodiments, each primer of the subset of primers bas at least 2 (such as 2, 3, 4, or more) phosphorothioate internucleotide bonds at the 3′ end of the primer. In other embodiments, the multiplexed PCR operation may amplify at least 100, 200, 300, 400, 500, 600, 700, 800, 900, 1000, or more target loci.

In some embodiments, the multiplex PCR method further includes treating the pool of primers with an enzyme having a 3′ to 5′ exonuclease activity prior to performing the multiplex PCR amplification. In some embodiments, the subset of primers corresponds to at least 50% of the primers in the pool of primers. In some embodiments, the method further comprises determining the amount of primer dimer formation following the multiplex PCR amplification. In some embodiments, the amount of primer dimer formation in the multiplex PCR amplification is less than 50%. In some embodiments, the amount of primer dimer formation in the multiplex PCR amplification is less than 70% of the amount of primer dimer formation in a corresponding multiplex PCR amplification where each of the primers in the pool of primers has only phosphodiester internucleotide bonds. In some embodiments, determining the amount of primer dimer formation is carried out by NGS.

In some embodiments, a method of amplifying at least 50 target loci in a nucleic acid sample includes performing multiplex PCR amplification on the nucleic acid sample using a pool of primers comprising a set of primer pairs collectively comprising at least one primer pair for each of the at least 50 target loci, wherein each primer of a subset of primers in the pool of primers comprises at least 2 phosphorothioate internucleotide bonds at the 3′ end of the primer including, but not limited to, the five 3′ terminal nucleotides. In some embodiments, each primer of the subset of primers comprises at least 3 (such as 3, 4, or more) phosphorothioate internucleotide bonds at the 3′ end of the primer. In some embodiments, the at least 50 target loci comprise at least 100 (such as at least any of 100, 200, 300, 400, 500, 600, 700, 800, 900, 1000, or more) target loci. In some embodiments, the method further comprises treating the pool of primers with an enzyme having a 3′ to 5′ exonuclease activity prior to performing the multiplex PCR amplification. In some embodiments, the subset of primers corresponds to at least 50% of the primers in the pool of primers. In some embodiments, the method further comprises determining the amount of primer dimer formation following the multiplex PCR amplification. In some embodiments, the amount of primer dimer formation in the multiplex PCR amplification is less than 50%. In some embodiments, the amount of primer dimer formation in the multiplex PCR amplification is less than 70% of the amount of primer dimer formation in a corresponding multiplex PCR amplification where each of the primers in the pool of primers has only phosphodiester internucleotide bonds. In some embodiments, determining the amount of primer dimer formation is carried out by NGS.

In some embodiments, a method of amplifying at least 100 target loci in a nucleic acid sample includes performing multiplex PCR amplification on the nucleic acid sample using a pool of primers comprising a set of primer pairs collectively comprising at least one primer pair for each of the at least 100 target loci, wherein each primer of a subset of primers in the pool of primers comprises at least 2 phosphorothioate internucleotide bonds at the 3′ end of the primer including, but not limited to, the five 3′ terminal nucleotides. In some embodiments, each primer of the subset of primers comprises at least 3 (such as 3, 4, or more) phosphorothioate internucleotide bonds at the 3′ end of the primer. In some embodiments, the at least 100 target loci comprise at least 200 (such as at least any of 200, 300, 400, 500, 600, 700, 800, 900, 1000, or more) target loci. In some embodiments, the method further comprises treating the pool of primers with an enzyme having a 3′ to 5′ exonuclease activity prior to performing the multiplex PCR amplification. In some embodiments, the subset of primers corresponds to at least 50% of the primers in the pool of primers. In some embodiments, the method further comprises determining the amount of primer dimer formation following the multiplex PCR amplification. In some embodiments, the amount of primer dimer formation in the multiplex PCR amplification is less than 50%. In some embodiments, the amount of primer dimer formation in the multiplex PCR amplification is less than 70% of the amount of primer dimer formation in a corresponding multiplex PCR amplification where each of the primers in the pool of primers has only phosphodiester internucleotide bonds. In some embodiments, determining the amount of primer dimer formation is carried out by NGS.

In some embodiments, a method of amplifying at least 50 target loci in a nucleic acid sample includes performing multiplex PCR amplification on the nucleic acid sample using a pool of primers comprising a set of primer pairs collectively comprising at least one primer pair for each of the at least 50 target loci, wherein each primer of a subset of primers in the pool of primers comprises a phosphorothioate internucleotide bond between the nucleotides at the 0 and −1 positions. In some embodiments, each primer of the subset of primers further comprises a phosphorothioate internucleotide bond between the nucleotides at the −1 and −2 positions. In some embodiments, each primer of the subset of primers further comprises a phosphorothioate internucleotide bond between the nucleotides at the −2 and −3 positions. In some embodiments, each primer of the subset of primers further comprises a phosphorothioate internucleotide bond between the nucleotides at the −3 and −4 positions. In some embodiments, the at least 50 target loci comprise at least 100 (such as at least any of 100, 200, 300, 400, 500, 600, 700, 800, 900, 1000, or more) target loci. In some embodiments, the method further comprises treating the pool of primers with an enzyme having a 3′ to 5′ exonuclease activity prior to performing the multiplex PCR amplification. In some embodiments, the subset of primers corresponds to at least 50% of the primers in the pool of primers. In some embodiments, the method further comprises determining the amount of primer dimer formation following the multiplex PCR amplification. In some embodiments, the amount of primer dimer formation in the multiplex PCR amplification is less than 50%. In some embodiments, the amount of primer dimer formation in the multiplex PCR amplification is less than 70% of the amount of primer dimer formation in a corresponding multiplex PCR amplification where each of the primers in the pool of primers has only phosphodiester internucleotide bonds. In some embodiments, determining the amount of primer dimer formation is carried out by NGS.

In some embodiments, a method of amplifying at least 100 target loci in a nucleic acid sample includes performing multiplex PCR amplification on the nucleic acid sample using a pool of primers comprising a set of primer pairs collectively comprising at least one primer pair for each of the at least 100 target loci, wherein each primer of a subset of primers in the pool of primers comprises a phosphorothioate internucleotide bond between the nucleotides at the 0 and −1 positions. In some embodiments, each primer of the subset of primers further comprises a phosphorothioate internucleotide bond between the nucleotides at the −1 and −2 positions. In some embodiments, each primer of the subset of primers further comprises a phosphorothioate internucleotide bond between the nucleotides at the −2 and −3 positions. In some embodiments, each primer of the subset of primers further comprises a phosphorothioate internucleotide bond between the nucleotides at the −3 and −4 positions. In some embodiments, the at least 100 target loci comprise at least 200 (such as at least any of 200, 300, 400, 500, 600, 700, 800, 900, 1000, or more) target loci. In some embodiments, the method further comprises treating the pool of primers with an enzyme having a 3′ to 5′ exonuclease activity prior to performing the multiplex PCR amplification. In some embodiments, the subset of primers corresponds to at least 50% of the primers in the pool of primers. In some embodiments, the method further comprises determining the amount of primer dimer formation following the multiplex PCR amplification. In some embodiments, the amount of primer dimer formation in the multiplex PCR amplification is less than 50%. In some embodiments, the amount of primer dimer formation in the multiplex PCR amplification is less than 70% of the amount of primer dimer formation in a corresponding multiplex PCR amplification where each of the primers in the pool of primers has only phosphodiester internucleotide bonds. In some embodiments, determining the amount of primer dimer formation is carried out by NGS.

In some embodiments a method of amplifying at least 50 target loci in a nucleic acid sample includes (a) performing multiplex PCR amplification on the nucleic acid sample using a pool of primers comprising a set of primer pairs collectively comprising at least one primer pair for each of the at least 50 target loci, wherein each primer of a subset of primers in the pool of primers comprises at least one exonuclease-resistant internucleotide bond at the 3′ end of the primer including, but not limited to, the five 3′ terminal nucleotides, thereby generating amplicons corresponding to the different primer pairs; and (b) processing the amplicons to be adapted for NGS. In some embodiments, the method further comprises carrying out NGS on the processed amplicons, thereby generating sequence read data for the amplicons. In some embodiments, the method further comprises determining sequence read depths for the amplicons based on the sequence read data. In some embodiments, the exonuclease-resistant internucleotide bond is a phosphorothioate bond, and each primer of the subset of primers comprises at least 2 (such as 2, 3, 4, or more) phosphorothioate internucleotide bonds at the 3′ end of the primer. In some embodiments, the at least 50 target loci comprise at least 100 (such as at least any of 100, 200, 300, 400, 500, 600, 700, 800, 900, 1000, or more) target loci. In some embodiments, the method further comprises treating the pool of primers with an enzyme having a 3′ to 5′ exonuclease activity prior to performing the multiplex PCR amplification. In some embodiments, the subset of primers corresponds to at least 50% of the primers in the pool of primers. In some embodiments, the method further comprises determining the amount of primer dimer formation following the multiplex PCR amplification. In some embodiments, the amount of primer dimer formation in the multiplex PCR amplification is less than 50%. In some embodiments, the amount of primer dimer formation in the multiplex PCR amplification is less than 70% of the amount of primer dimer formation in a corresponding multiplex PCR amplification where each of the primers in the pool of primers has only phosphodiester internucleotide bonds. In some embodiments, determining the amount of primer dimer formation is carried out by NGS.

### Exonuclease-Resistant Nucleotide

In another aspect, a method of amplifying a plurality of target loci in a nucleic acid sample includes performing multiplex PCR amplification on the nucleic acid sample using a pool of primers comprising a set of primer pairs collectively comprising at least one primer pair for each of the plurality of target loci, wherein each primer of a subset of primers in the pool of primers comprises at least one exonuclease-resistant nucleotide. In some embodiments, the plurality of target loci comprises at least 50 (such as at least any of 50, 100, 200, 300, 400, 500, 600, 700, 800, 900, 1000, or more) target loci. In some embodiments, the at least one exonuclease-resistant nucleotide in a primer is present at the 3′ end of the primer. In this context, the 3′ end of a primer includes, but is not limited to, the five 3′ terminal nucleotides. In some embodiments, the at least one exonuclease-resistant nucleotide comprises a plurality of (such as any of 2, 3, 4, 5, or more) exonuclease-resistant nucleotides.

In some embodiments, each primer of the subset of primers comprises an exonuclease-resistant nucleotide at the 0, −1, −2, or −3 positions. For example, in some embodiments, a primer of the subset of primers comprises an exonuclease-resistant nucleotide at the 0 position. In some embodiments, a primer of the subset of primers comprises an exonuclease-resistant nucleotide at the −1 position. In some embodiments, a primer of the subset of primers comprises an exonuclease-resistant nucleotide at the −2 position. In some embodiments, a primer of the subset of primers comprises an exonuclease-resistant nucleotide at the −3 position.

In some embodiments, the exonuclease-resistant nucleotide is a locked nucleic acid (LNA) nucleotide. In some embodiments, the LNA nucleotide is a nucleotide having a sugar moiety with a bridge connecting the 2′ oxygen and 4′ carbon.

In some embodiments, each primer of the subset of primers comprises an LNA nucleotide at the 0, −1, −2, or −3 positions. In some embodiments, a primer of the subset of primers comprises 1, 2, 3, or 4 LNA nucleotides. For example, in some embodiments, a primer of the subset of primers comprises an LNA nucleotide at the 0 position. In some embodiments, a primer of the subset of primers comprises an LNA nucleotide at the −1 position. In some embodiments, a primer of the subset of primers comprises an LNA nucleotide at the −2 position. In some embodiments, a primer of the subset of primers comprises an LNA nucleotide at the −3 position. In some embodiments, a primer of the subset of primers comprises LNA nucleotides at the 0 and −1 positions. In some embodiments, a primer of the subset of primers comprises LNA nucleotides at the 0, −1, and −2 positions. In some embodiments, a primer of the subset of primers comprises LNA nucleotides at the 0, −1, −2, and −3 positions.

In some embodiments, the method further comprises treating the pool of primers with an enzyme having a 3′ to 5′ exonuclease activity prior to performing the multiplex PCR amplification. In some embodiments, the pool of primers are treated with the enzyme having a 3′ to 5′ exonuclease activity under conditions that allow for at least 50% (such at least any of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) of primers in the pool of primers having no exonuclease-resistant nucleotides to be digested. In some embodiments, following enzymatic treatment, less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%) of the primers remaining from the pool of primers do not comprise at least one exonuclease-resistant nucleotide. In some embodiments, the enzyme-treated primers are purified prior to use in the multiple PCR amplification. In some embodiments, the enzyme-treated primers are used in the multiple PCR amplification following inactivation (e.g., heat inactivation) of the enzyme. In some embodiments, the enzyme having a 3′ to 5′ exonuclease activity is, inter alia, a DNA polymerase I, a T7 DNA polymerase, a Klenow fragment, a vent DNA polymerase, or a Pfu DNA polymerase.

In some embodiments, the subset of primers corresponds to at least 50% (such at least any of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) of the primers in the pool of primers.

In some embodiments, the method further comprises determining the amount of primer dimer formation following the multiplex PCR amplification. In some embodiments, the amount of primer dimer formation in the multiplex PCR amplification is less than 50% (such as less than any of 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amount of primer dimer formation in the multiplex PCR amplification is less than 70% (such as less than any of 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) of the amount of primer dimer formation in a corresponding multiplex PCR amplification where each of the primers in the pool of primers has only natural nucleotides. In some embodiments, determining the amount of primer dimer formation is carried out by next generation sequencing (NGS). For example, once the multiplex PCR amplification has been carried out, the resulting amplicons can be sequenced using NGS technology, and sequencing reads can be mapped back to the sequences of primers from the pool of primers. If primer dimers have formed, they will be represented as sequencing reads in the NGS data. These reads can be counted or quantified to determine the extent of primer dimer formation. In some alternative embodiments, determining the amount of primer dimer formation is carried out by gel electrophoresis or high-resolution melting analysis (HRMA).

In some embodiments, the method further comprises determining the amount of primer dimer formation following the multiplex PCR amplification for a subset of primers in the pool of primers, and generating a filtered set of primer pairs based on the set of primer pairs in the pool of primers that excludes, for each primer dimer found to be present above a predetermined threshold, at least one of the primer pairs comprising a primer in the corresponding primer dimer. In some embodiments, the predetermined threshold is 1/N, where N is the number of primer pairs. In other words, if a primer dimer is getting as many reads as would be expected for a target amplicon (assuming the target read depth is the same for all target amplicons) then one or both of the primers forming the primer dimer are removed for the next iteration of the load-balancing process. It should be appreciated that other thresholds may be used and that the threshold may be dynamically determined based on the sequence read results.

In some embodiments, the method further comprises processing amplicons generated in the multiplex PCR amplification to be adapted for NGS. In some embodiments, the method further comprises carrying out NGS on the processed amplicons, thereby generating sequence read data for the amplicons. In some embodiments, the method further comprises determining sequence read depths for the amplicons based on the sequence read data.

In some embodiments, the method of amplifying a plurality of target loci employs a subset of primers comprising an exonuclease-resistant nucleotide, the method further comprises using the multiplex PCR amplification in a downstream application (e.g., a diagnostic or other assay).

In some embodiments, a method comprises performing multiplex PCR amplification on a nucleic acid sample having at least 50 target loci using a pool of primers comprising a set of primer pairs collectively comprising at least one primer pair for each of the at least 50 target loci, wherein each primer of a subset of primers in the pool of primers comprises at least one exonuclease-resistant nucleotide at the 3′ end of the primer, such as the five 3′ terminal nucleotides. In some embodiments, the exonuclease-resistant nucleotide is an LNA nucleotide, and each primer of the subset of primers comprises at least 2 (such as 2, 3, 4, or more) LNA nucleotides at the 3′ end of the primer. In some embodiments, the target loci comprise at least 100 (such as at least any of 100, 200, 300, 400, 500, 600, 700, 800, 900, 1000, or more) target loci. In some embodiments, the method further comprises treating the pool of primers with an enzyme having a 3′ to 5′ exonuclease activity prior to performing the multiplex PCR amplification. In some embodiments, the subset of primers corresponds to at least 50% of the primers in the pool of primers. In some embodiments, the method further comprises determining the amount of primer dimer formation following the multiplex PCR amplification. In some embodiments, the amount of primer dimer formation in the multiplex PCR amplification is less than 50%. In some embodiments, the amount of primer dimer formation in the multiplex PCR amplification is less than 70% of the amount of primer dimer formation in a corresponding multiplex PCR amplification where each of the primers in the pool of primers has only natural nucleotides. In some embodiments, determining the amount of primer dimer formation is carried out by NGS.

In some embodiments, the method of amplifying at least 50 target loci in a nucleic acid sample comprises performing multiplex PCR amplification on the nucleic acid sample using a pool of primers comprising a set of primer pairs collectively comprising at least one primer pair for each of the at least 50 target loci, wherein each primer of a subset of primers in the pool of primers comprises at least one LNA nucleotide at the 3′ end of the primer, such as the five 3′ terminal nucleotides. In some embodiments, each primer of the subset of primers comprises at least 2 (such as 2, 3, 4, or more) LNA nucleotides at the 3′ end of the primer. In some embodiments, the at least 50 target loci comprise at least 100 (such as at least any of 100, 200, 300, 400, 500, 600, 700, 800, 900, 1000, or more) target loci. In some embodiments, the method further comprises treating the pool of primers with an enzyme having a 3′ to 5′ exonuclease activity prior to performing the multiplex PCR amplification. In some embodiments, the subset of primers corresponds to at least 50% of the primers in the pool of primers. In some embodiments, the method further comprises determining the amount of primer dimer formation following the multiplex PCR amplification. In some embodiments, the amount of primer dimer formation in the multiplex PCR amplification is less than 50%. In some embodiments, the amount of primer dimer formation in the multiplex PCR amplification is less than 70% of the amount of primer dimer formation in a corresponding multiplex PCR amplification where each of the primers in the pool of primers has only natural nucleotides. In some embodiments, determining the amount of primer dimer formation is carried out by NGS.

In some embodiments, a method of amplifying at least 100 target loci in a nucleic acid sample comprises performing multiplex PCR amplification on a nucleic acid sample having at least 100 target loci using a pool of primers comprising a set of primer pairs collectively comprising at least one primer pair for each of the at least 100 target loci, wherein each primer of a subset of primers in the pool of primers comprises at least 1 LNA nucleotide at the 3′ end of the primer, such as the five 3′ terminal nucleotides. In some embodiments, each primer of the subset of primers comprises at least 2 (such as 2, 3, 4, or more) LNA nucleotides at the 3′ end of the primer. In some embodiments, the at least 100 target loci comprise at least 200 (such as at least any of 200, 300, 400, 500, 600, 700, 800, 900, 1000, or more) target loci. In some embodiments, the method further comprises treating the pool of primers with an enzyme having a 3′ to 5′ exonuclease activity prior to performing the multiplex PCR amplification. In some embodiments, the subset of primers corresponds to at least 50% of the primers in the pool of primers. In some embodiments, the method further comprises determining the amount of primer dimer formation following the multiplex PCR amplification. In some embodiments, the amount of primer dimer formation in the multiplex PCR amplification is less than 50%. In some embodiments, the amount of primer dimer formation in the multiplex PCR amplification is less than 70% of the amount of primer dimer formation in a corresponding multiplex PCR amplification where each of the primers in the pool of primers has only natural nucleotides. In some embodiments, determining the amount of primer dimer formation is carried out by NGS.

In some embodiments, a method of amplifying at least 50 target loci in a nucleic acid sample comprises performing multiplex PCR amplification on the nucleic acid sample using a pool of primers comprising a set of primer pairs collectively comprising at least one primer pair for each of the at least 50 target loci, wherein each primer of a subset of primers in the pool of primers comprises an LNA nucleotide at the 0 position. In some embodiments, each primer of the subset of primers further comprises an LNA nucleotide at the −1 position. In some embodiments, each primer of the subset of primers further comprises an LNA nucleotide at the −2 position. In some embodiments, each primer of the subset of primers further comprises an LNA nucleotide at the −3 position. In some embodiments, the at least 50 target loci comprise at least 100 (such as at least any of 100, 200, 300, 400, 500, 600, 700, 800, 900, 1000, or more) target loci. In some embodiments, the method further comprises treating the pool of primers with an enzyme having a 3′ to 5′ exonuclease activity prior to performing the multiplex PCR amplification. In some embodiments, the subset of primers corresponds to at least 50% of the primers in the pool of primers. In some embodiments, the method further comprises determining the amount of primer dimer formation following the multiplex PCR amplification. In some embodiments, the amount of primer dimer formation in the multiplex PCR amplification is less than 50%. In some embodiments, the amount of primer dimer formation in the multiplex PCR amplification is less than 70% of the amount of primer dimer formation in a corresponding multiplex PCR amplification where each of the primers in the pool of primers has only natural nucleotides. In some embodiments, determining the amount of primer dimer formation is carried out by NGS.

In some embodiments, a method of amplifying at least 100 target loci in a nucleic acid sample comprises performing multiplex PCR amplification on the nucleic acid sample using a pool of primers comprising a set of primer pairs collectively comprising at least one primer pair for each of the at least 100 target loci, wherein each primer of a subset of primers in the pool of primers comprises a LNA nucleotide at the 0 position. In some embodiments, each primer of the subset of primers further comprises a LNA nucleotide at the −1 position. In some embodiments, each primer of the subset of primers further comprises a LNA nucleotide at the −2 position. In some embodiments, each primer of the subset of primers further comprises a LNA nucleotide at the −3 position. In some embodiments, the at least 100 target loci comprise at least 200 (such as at least any of 200, 300, 400, 500, 600, 700, 800, 900, 1000, or more) target loci. In some embodiments, the method further comprises treating the pool of primers with an enzyme having a 3′ to 5′ exonuclease activity prior to performing the multiplex PCR amplification. In some embodiments, the subset of primers corresponds to at least 50% of the primers in the pool of primers. In some embodiments, the method further comprises determining the amount of primer dimer formation following the multiplex PCR amplification. In some embodiments, the amount of primer dimer formation in the multiplex PCR amplification is less than 50%. In some embodiments, the amount of primer dimer formation in the multiplex PCR amplification is less than 70% of the amount of primer dimer formation in a corresponding multiplex PCR amplification where each of the primers in the pool of primers has only natural nucleotides. In some embodiments, determining the amount of primer dimer formation is carried out by NGS.

In some embodiments, a method of amplifying at least 50 target loci in a nucleic acid sample comprises performing multiplex PCR amplification on the nucleic acid sample using a pool of primers comprising a set of primer pairs collectively comprising at least one primer pair for each of the at least 50 target loci, wherein each primer of a subset of primers in the pool of primers comprises at least one exonuclease-resistant nucleotide at the 3′ end of the primer including, but not limited to, the five 3′ terminal nucleotides, thereby generating amplicons corresponding to the different primer pairs; and (b) processing the amplicons to be adapted for NGS. In some embodiments, the method further comprises carrying out NGS on the processed amplicons, thereby generating sequence read data for the amplicons In some embodiments, the method further comprises determining sequence read depths for the amplicons based on the sequence read data. In some embodiments, the exonuclease-resistant nucleotide is an LNA nucleotide, and each primer of the subset of primers comprises at least 2 (such as 2, 3, 4, or more) LNA nucleotides at the 3′ end of the primer. In some embodiments, the at least 50 target loci comprise at least 100 (such as at least any of 100, 200, 300, 400, 500, 600, 700, 800, 900, 1000, or more) target loci. In some embodiments, the method further comprises treating the pool of primers with an enzyme having a 3′ to 5′ exonuclease activity prior to performing the multiplex PCR amplification. In some embodiments, the subset of primers corresponds to at least 50% of the primers in the pool of primers. In some embodiments, the method further comprises determining the amount of primer dimer formation following the multiplex PCR amplification. In some embodiments, the amount of primer dimer formation in the multiplex PCR amplification is less than 50%. In some embodiments, the amount of primer dimer formation in the multiplex PCR amplification is less than 70% of the amount of primer dimer formation in a corresponding multiplex PCR amplification where each of the primers in the pool of primers has only natural nucleotides. In some embodiments, determining the amount of primer dimer formation is carried out by NGS.

### Additional Embodiments

The described approaches for addressing primer-dimer formation may be used together in any combination. In some embodiments, exonuclease-resistant internucleotide bonds and exonuclease-resistant nucleotides are used together. This can address two potentially competing goals in a multiplex PCR amplification: (i) reducing the amount of primer dimers generated by the multiplex PCR; and (ii) maintaining a uniform read balance in which each amplicon generates approximately the same number (or a corresponding target number) of sequencing reads. Incorporating PTO bonds to the 3′ end of a primer makes it resistant to the exonuclease of the DNA polymerase, reducing the formation of primer dimers. However, incorporating PTO bonds can decrease the annealing temperature of the primer with its complementary DNA target, decreasing the number of reads for that amplicon. Conversely, a LNA in a primer can increase the annealing temperature of the primer with its target. Thus, incorporating one or more LNAs into a primer in addition to one or more PTO bonds can increase the read depth of that amplicon, give it exonuclease resistant properties, and balance the impact on annealing temperature of the PTO bonds and LNAs. For example, the PTO bonds can be at the 3′ end and the LNA can be in the 5′ end of the primer.

In view of this, some embodiments of a method of amplifying a plurality of target loci in a nucleic acid sample comprises performing multiplex PCR amplification on the nucleic acid sample using a pool of primers having at least one primer pair for each of the plurality of target loci. Each primer of a subset of primers in the pool of primers has at least one exonuclease-resistant internucleotide bond and at least one exonuclease-resistant nucleotide. In some embodiments, the plurality of target loci comprises at least 50 (such as at least any of 50, 100, 200, 300, 400, 500, 600, 700, 800, 900, 1000, or more) target loci. In some embodiments, the at least one exonuclease-resistant internucleotide bond and the at least one exonuclease-resistant nucleotide in a primer is present at the 3′ end of the primer. In this context, the 3′ end of a primer includes, but is not limited to, the five 3′ terminal nucleotides. In some embodiments, the at least one exonuclease-resistant internucleotide bond comprises a plurality of (such as any of 2, 3, 4, 5, or more) exonuclease-resistant internucleotide bonds. In some embodiments, the at least one exonuclease-resistant nucleotide comprises a plurality of (such as any of 2, 3, 4, 5, or more) exonuclease-resistant nucleotides. In some embodiments, the exonuclease-resistant internucleotide bond is a phosphorothioate internucleotide bond. In some embodiments, the exonuclease-resistant nucleotide is an LNA nucleotide.

In some embodiments, the method further comprises treating the pool of primers with an enzyme having a 3′ to 5′ exonuclease activity prior to performing the multiplex PCR amplification. In some embodiments, the pool of primers are treated with the enzyme having a 3′ to 5′ exonuclease activity under conditions that allow for at least 50% (such at least any of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) of primers in the pool of primers having no exonuclease-resistant internucleotide bonds or exonuclease-resistant nucleotides to be digested. In some embodiments, following enzymatic treatment, less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%) of the primers remaining from the pool of primers do not comprise at least one exonuclease-resistant internucleotide bond and at least one exonuclease-resistant nucleotide. In some embodiments, the enzyme-treated primers are purified prior to use in the multiple PCR amplification. In some embodiments, the enzyme-treated primers are used in the multiple PCR amplification following inactivation (e.g., heat inactivation) of the enzyme. In some embodiments, the enzyme having a 3′ to 5′ exonuclease activity is, inter alia, a DNA polymerase I, a T7 DNA polymerase, a Klenow fragment, a vent DNA polymerase, or a Pfu DNA polymerase.

In some embodiments, the subset of primers corresponds to at least 50% (such at least any of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) of the primers in the pool of primers.

In some embodiments, the method further comprises determining the amount of primer dimer formation following the multiplex PCR amplification. In some embodiments, the amount of primer dimer formation in the multiplex PCR amplification is less than 50% (such as less than any of 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amount of primer dimer formation in the multiplex PCR amplification is less than 70% (such as less than any of 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) of the amount of primer dimer formation in a corresponding multiplex PCR amplification where each of the primers in the pool of primers has only phosphodiester internucleotide bonds and natural nucleotides. In some embodiments, determining the amount of primer dimer formation is carried out by next generation sequencing (NGS). For example, once the multiplex PCR amplification has been carried out, the resulting amplicons can be sequenced using NGS technology, and sequencing reads can be mapped back to the sequences of primers from the pool of primers. If primer dimers have formed, they will be represented as sequencing reads in the NGS data. These reads can be counted or quantified to determine the extent of primer dimer formation. In some alternative embodiments, determining the amount of primer dimer formation is carried out by gel electrophoresis or high-resolution melting analysis (HRMA).

### Methods of Multiplex PCR Load Balancing

The present disclosure also provides methods of performing multiplex PCR amplification on a nucleic acid sample where the concentrations of the primers used have been adjusted such that the read depth of amplicons from the multiplex PCR are closer to target read depths for the amplicons than they would be without the primer concentration adjustment. As discussed above, one technical challenge with performing multiplex PCR is variance in amplicon read depth across primer pairs arising from differences in amplification efficiency and second-order effects between different primer pairs. Balancing the concentration of primers used in a multiplex PCR (also referred to as “load balancing”) based on the read depths achieved in one or more previous multiplex PCR reactions can provide a target read depth for each amplicon within a predetermined threshold of acceptable error. For example, the load balancing can limit the variance in read depth across amplicons, facilitating downstream analysis employing such parameters. Alternatively, the load balancing can focus amplification on amplicons where a greater read depth is needed for downstream analysis, such as amplicons where the expected signal to noise ratio for an assay is small.

### Two-Part Methods

In some instances, a two-part method is sufficient to obtain a primer pair concentration profile that meets the target read depth requirements of the use case within an acceptable error threshold. In such instances, an initial multiplex PCR amplification is performed using initial primer pair concentrations and the resulting sequence read depths for the amplicons is determined. The concentrations of the primer pairs are then modified based on a comparison of the read depths obtained and the target read depths to produce a primer concentration profile that provides approximately the target read depths. For example, the modified concentration profile may result in read depths for each amplicon that is within 1%, 5%, 10%, or 20%, etc., of a corresponding target read depth.

In some embodiments, a method of amplifying a plurality of target loci in a nucleic acid sample comprises (a) generating or obtaining a first set of primer pairs collectively comprising at least one primer pair for each of the plurality of target loci; (b) performing a first multiplex PCR amplification on a first nucleic acid sample using the first set of primer pairs, wherein at least a subset of the first set of primer pairs are used in the first multiplex PCR amplification at concentrations according to a first primer pair concentration profile, thereby generating amplicons corresponding to the different primer pairs; (c) determining sequence read depths of amplicons from the first multiplex PCR amplification corresponding to the different primer pairs to generate a first amplification profile; (d) generating a second primer pair concentration profile based on the first amplification profile; and (e) performing a second multiplex PCR amplification on a second nucleic acid sample using a second set of primer pairs selected from the first set of primer pairs, wherein at least a subset of the second set of primer pairs is used in the second multiplex PCR amplification at concentrations according to the second primer pair concentration profile, thereby generating amplicons corresponding to the different primer pairs, wherein the concentration of each primer pair in the second primer pair concentration profile is selected based on the first amplification profile to bring the read depth for the corresponding amplicon resulting from the second multiplex PCR amplification closer to a target read depth for the corresponding amplicon. Such a second primer pair concentration profile is also referred to herein as a load-balanced primer pair concentration profile. In some embodiments, the plurality of target loci comprises at least 50 (such as at least any of 50, 100, 200, 300, 400, 500, 600, 700, 800, 900, 1000, or more) target loci. In some embodiments, the plurality of target loci comprises at least 100 target loci. In some embodiments, the plurality of target loci comprises at least 1000 target loci.

In some embodiments, the first multiplex PCR amplification comprises a plurality of reactions, wherein for each reaction, each primer pair of the at least a subset of the first set of primer pairs is used, independently, in the reaction at one of a plurality of (such as at least 2, 3, 4, 5, 6, 7, 8, 9, 10, 11, 12, 13, 14, 15, or more) concentrations according to the first primer pair concentration profile. In some embodiments, the plurality of reactions collectively comprises each primer pair of the at least a subset of the first set of primer pairs at each concentration of the respective plurality of concentrations. For example, in some embodiments, where the first set of primer pairs comprises primer pairs A and B, and the first primer pair concentration profile comprises primer pair A at concentrations [A]₁ and [A]₂ and primer pair B at concentrations [B]₁ and [B]₂, the plurality of reactions can comprise (a) a first reaction with primer pairs A and B at concentrations [A]₁ and [B]₁, respectively, and a second reaction with primer pairs A and B at concentrations [A]₂ and [B]₂, respectively, or (b) a first reaction with primer pairs A and B at concentrations [A]₁ and [B]₂, respectively, and a second reaction with primer pairs A and B at concentrations [A]₂ and [B]₁, respectively. The plurality of concentrations for each primer pair in the first primer pair concentration profile need not be the same. For example, in some embodiments, where the first set of primer pairs comprises primer pairs A and B, the first primer pair concentration profile can comprise A at concentrations [A]₁ and [A]₂ and primer pair B at concentrations [B]₁-[B]₄, wherein neither [A]₁ nor [A]₂ are equal to any of [B]₁-[B]₄. Various combinations of the number of concentrations and their respective values are contemplated, and are not limited by the examples provided herein, which are presented for illustrative purposes.

In some embodiments, the first primer pair concentration profile comprises, independently, each primer pair of the at least a subset of the first set of primer pairs at a plurality of (such as at least 2, 3, 4, 5, 6, 7, 8, 9, 10, 11, 12, 13, 14, 15, or more) concentrations. In some embodiments, the plurality of concentrations collectively comprise concentrations ranging from between 100 pM and 10 pM (such as any of 100 pM, 150 pM, 200 pM, 250 pM, 300 pM, 350 pM, 400 pM, 450 pM, 500 pM, 550 pM, 600 pM, 650 pM, 700 pM, 750 pM, 800 pM, 850 pM, 900 pM, 950 pM, 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 220 nM, 240 nM, 260 nM, 280 nM, 300 nM, 320 nM, 340 nM, 360 nM, 380 nM, 400 nM, 420 nM, 440 nM, 460 nM, 480 nM, 500 nM, 520 nM, 540 nM, 560 nM, 580 nM, 600 nM, 620 nM, 640 nM, 660 nM, 680 nM, 700 nM, 720 nM, 740 nM, 760 nM, 780 nM, 800 nM, 820 nM, 840 nM, 860 nM, 880 nM, 900 nM, 920 nM, 940 nM, 960 nM, 980 nM, 1 μM, 2 μM, 3 μM, 4 μM, 5 μM, 6 μM, 7 μM, 8 μM, 9 μM, or 10 μM, including any ranges between any of these values). In some embodiments, determining sequence read depths of amplicons from the first multiplex PCR amplification comprises determining sequence read depths of amplicons corresponding to each primer pair of the at least a subset of the first set of primer pairs at each of the respective plurality of concentrations to generate the first amplification profile. In some embodiments, the method further comprises using the first amplification profile to determine a relationship between primer pair concentration and sequence read depth for each primer pair of the at least a subset of the first set of primer pairs. In some embodiments, the relationship between primer pair concentration, or [P], and sequence read depth, or reads, is modeled as reads=b (2−a^{-[P]})²⁰. In some embodiments, the model is fit using a nonlinear least-squares fit. In some embodiments, the fit is performed in the log-log space. In some embodiments, generating the second primer pair concentration profile based on the first amplification profile comprises using the relationship between primer pair concentration and sequence read depth for each primer pair of the at least a subset of the first set of primer pairs to determine the concentration for each primer pair of the at least a subset of the first set of primer pairs corresponding to a target sequence read depth. Any suitable target sequence read depth can be selected, and this can vary depending on downstream applications of the multiplex PCR amplification.

In some embodiments, NGS is used to determine the sequence read depths of amplicons. For example, in some embodiments, the method further comprises steps of (i) processing a set of amplicons from a multiplex PCR amplification to be adapted for NGS. (ii) carrying out NGS on the processed amplicons, thereby generating sequence read data for the set of amplicons, and (iii) determining sequence read depths for the set of amplicons based on the sequence read data.

In some embodiments, the highest and lowest amplicon read depths from the first multiplex PCR amplification vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). For example, in some embodiments, the highest and lowest amplicon read depths from the first multiplex PCR amplification vary by at least 5-fold. In some embodiments, the highest and lowest amplicon read depths from the first multiplex PCR amplification vary by at least 10-fold. In some embodiments, the highest and lowest amplicon read depths from the first multiplex PCR amplification vary by at least 100-fold. In some embodiments, the highest and lowest amplicon read depths from the first multiplex PCR amplification vary by at least 1000-fold.

In some embodiments, the highest and lowest primer pair concentrations associated with the second primer pair concentration profile vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). For example, in some embodiments, the highest and lowest primer pair concentrations associated with the second primer pair concentration profile vary by at least 5-fold. In some embodiments, the highest and lowest primer pair concentrations associated with the second primer pair concentration profile vary by at least 10-fold. In some embodiments, the highest and lowest primer pair concentrations associated with the second primer pair concentration profile vary by at least 100-fold. In some embodiments, the highest and lowest primer pair concentrations associated with the second primer pair concentration profile vary by at least 1000-fold.

In some embodiments, the method further comprises determining sequence read depths of amplicons from the second multiplex PCR amplification, wherein the amplicon read depths from the second multiplex PCR amplification have a coefficient of variance (CV) of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). For example, in some embodiments, the amplicon read depths from the second multiplex PCR amplification corresponding to the different primer pairs have a CV of less than 10%. In some embodiments, the amplicon read depths from the second multiplex PCR amplification corresponding to the different primer pairs have a CV of less than 5%. In some embodiments, the amplicon read depths from the second multiplex PCR amplification corresponding to the different primer pairs have a CV of less than 2%. In some embodiments, the amplicon read depths from the second multiplex PCR amplification corresponding to the different primer pairs have a CV of less than 1%.

In some embodiments, the method further comprises determining sequence read depths of amplicons from the second multiplex PCR amplification, wherein the amplicon read depths from the second multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less). For example, in some embodiments, the amplicon read depths from the second multiplex PCR amplification corresponding to the different primer pairs have a 90^{th} percentile over 10^{th} percentile of less than 1.4. In some embodiments, the amplicon read depths from the second multiplex PCR amplification corresponding to the different primer pairs have a 90^{th} percentile over 10^{th} percentile of less than 1.25. In some embodiments, the amplicon read depths from the second multiplex PCR amplification corresponding to the different primer pairs have a 90^{th} percentile over 10^{th} percentile of less than 1.1. In some embodiments, the amplicon read depths from the second multiplex PCR amplification corresponding to the different primer pairs have a 90^{th} percentile over 10^{th} percentile of less than 1.05.

In some embodiments, the method further comprises determining the amount of primer dimer formation (such as by NGS) following the first multiplex PCR amplification for at least a subset of primers used in the first multiplex PCR amplification, and generating a filtered set of primer pairs based on the first set of primer pairs that excludes, for each primer dimer found to be present above a predetermined threshold, at least one of the primer pairs comprising a primer in the corresponding primer dimer. In some embodiments, the filtered set of primer pairs is used in the second multiplex PCR amplification. In some embodiments, the predetermined threshold is 1/N, where N is the number of primer pairs.

In some embodiments, the method further comprises determining the amount of primer dimer formation (such as by NGS) following the second multiplex PCR amplification for at least a subset of primers used in the second multiplex PCR amplification, and generating a filtered set of primer pairs based on the second set of primer pairs that excludes, for each primer dimer found to be present above a predetermined threshold, at least one of the primer pairs comprising a primer in the corresponding primer dimer. In some embodiments, the predetermined threshold is 1/N, where N is the number of primer pairs.

In some embodiments, for the first or second multiplex PCR amplification, independently, each primer of at least a subset of primers used in the respective multiplex PCR amplification comprises at least one exonuclease-resistant internucleotide bond or at least one exonuclease-resistant nucleotide according to any of the embodiments described herein. In some embodiments, one or more primers of the at least a subset of primers comprises at least one exonuclease-resistant internucleotide bond at the 3′ end of the primer including, but not limited to, the five 3′ terminal nucleotides. In some embodiments, the exonuclease-resistant internucleotide bond is a phosphorothioate bond. In some embodiments, each primer of the one or more primers comprises at least 2 (such as 2, 3, 4, or more) phosphorothioate internucleotide bonds at the 3′ end of the primer. In some embodiments, each primer of the one or more primers comprises a phosphorothioate internucleotide bond between the nucleotide at the 0 position and the nucleotide at the −1 position and one or more phosphorothioate internucleotide bonds between any of (a) the nucleotide at the −1 position and the nucleotide at the −2 position; (b) the nucleotide at the −2 position and the nucleotide at the −3 position; and (c) the nucleotide at the −3 position and the nucleotide at the −4 position. In some embodiments, one or more primers of the at least a subset of primers comprises at least one exonuclease-resistant nucleotide at the 3′ end of the primer including, but not limited to, the five 3′ terminal nucleotides. In some embodiments, the exonuclease-resistant nucleotide is a locked nucleic acid (LNA) nucleotide, such as a nucleotide having a sugar moiety with a bridge connecting the 2′ oxygen and 4′ carbon. In some embodiments, each primer of the one or more primers comprises at least 2 (such as 2, 3, 4, or more) LNA nucleotides at the 3′ end of the primer. In some embodiments, each primer of the one or more primers comprises an LNA nucleotide at the 0 position and one or more LNA nucleotides at any of the −1, −2, and −3 positions. In some embodiments, the at least a subset of primers corresponds to at least 50% of the primers used in the respective multiplex PCR amplification.

In some embodiments, a method of amplifying at least 50 target loci in a nucleic acid sample comprises (a) generating or obtaining a first set of primer pairs collectively comprising at least one primer pair for each of the at least 50 target loci; (b) performing a first multiplex PCR amplification on a first nucleic acid sample using the first set of primer pairs, wherein at least a subset of the first set of primer pairs are used in the first multiplex PCR amplification at concentrations according to a first primer pair concentration profile, thereby generating amplicons corresponding to the different primer pairs; (c) determining sequence read depths of amplicons from the first multiplex PCR amplification corresponding to the different primer pairs to generate a first amplification profile; (d) generating a second primer pair concentration profile based on the first amplification profile; and (e) performing a second multiplex PCR amplification on a second nucleic acid sample using a second set of primer pairs selected from the first set of primer pairs, wherein at least a subset of the second set of primer pairs is used in the second multiplex PCR amplification at concentrations according to the second primer pair concentration profile, thereby generating amplicons corresponding to the different primer pairs, wherein the concentration of each primer pair in the second primer pair concentration profile is selected based on the first amplification profile to obtain amplicon read depths from the second multiplex PCR amplification that are closer to target read depths corresponding to the different primer pairs. In some embodiments, determining sequence read depths of amplicons is carried out by NGS.

In some embodiments, the first multiplex PCR amplification comprises a plurality of reactions, wherein for each reaction, each primer pair of the at least a subset of the first set of primer pairs is used, independently, in the reaction at one of a plurality of (such as at least 2, 3, 4, 5, 6, 7, 8, 9, 10, 11, 12, 13, 14, 15, or more) concentrations according to the first primer pair concentration profile. In some embodiments, the highest and lowest amplicon read depths from the first multiplex PCR amplification vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). In some embodiments, the highest and lowest primer pair concentrations associated with the second primer pair concentration profile vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more).

In some embodiments, the method further comprises determining sequence read depths of amplicons from the second multiplex PCR amplification (e.g., by NGS). In some embodiments, the amplicon read depths from the second multiplex PCR amplification have a CV of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amplicon read depths from the second multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less). In some embodiments, the method further comprises determining the amount of primer dimer formation (such as by NGS) following the first multiplex PCR amplification for at least a subset of primers used in the first multiplex PCR amplification, and generating a filtered set of primer pairs based on the first set of primer pairs that excludes, for each primer dimer found to be present above a predetermined threshold, at least one of the primer pairs comprising a primer in the corresponding primer dimer. In some embodiments, for the first or second multiplex PCR amplification, independently, each primer of at least a subset of primers used in the respective multiplex PCR amplification comprises at least one exonuclease-resistant internucleotide bond or at least one exonuclease-resistant nucleotide according to any of the embodiments described herein.

In some embodiments, the at least 50 target loci comprises at least 100 (such as at least any of 100, 200, 300, 400, 500, 600, 700, 800, 900, 1000, or more) target loci. In some embodiments, the at least 50 target loci comprises at least 500 target loci. In some embodiments, the at least 50 target loci comprises at least 1000 target loci.

In some embodiments, a method of amplifying at least 50 target loci in a nucleic acid sample comprises (a) generating or obtaining a first set of primer pairs collectively comprising at least one primer pair for each of the at least 50 target loci; (b) performing a first multiplex PCR amplification on a first nucleic acid sample using the first set of primer pairs, wherein at least a subset of the first set of primer pairs are used in the first multiplex PCR amplification at concentrations according to a first primer pair concentration profile, thereby generating amplicons corresponding to the different primer pairs, (c) determining, by NGS, sequence read depths of amplicons from the first multiplex PCR amplification corresponding to the different primer pairs to generate a first amplification profile; (d) generating a second primer pair concentration profile based on the first amplification profile; and (e) performing a second multiplex PCR amplification on a second nucleic acid sample using a second set of primer pairs selected from the first set of primer pairs, wherein at least a subset of the second set of primer pairs is used in the second multiplex PCR amplification at concentrations according to the second primer pair concentration profile, thereby generating amplicons corresponding to the different primer pairs, wherein the concentration of each primer pair in the second primer pair concentration profile is selected based on the first amplification profile to produce amplicon read depths from the second multiplex PCR amplification that are closer to target read depths corresponding to the different primer pairs. In some embodiments, the method further comprises (i) processing a set of amplicons from a multiplex PCR amplification to be adapted for NGS and (ii) carrying out NGS on the processed amplicons, thereby generating sequence read data for the set of amplicons. In some embodiments, the method further comprises determining sequence read depths for the set of amplicons based on the sequence read data.

In some embodiments, the first multiplex PCR amplification comprises a plurality of reactions, wherein for each reaction, each primer pair of the at least a subset of the first set of primer pairs is used, independently, in the reaction at one of a plurality of (such as at least 2, 3, 4, 5, 6, 7, 8, 9, 10, 11, 12, 13, 14, 15, or more) concentrations according to the first primer pair concentration profile. In some embodiments, the highest and lowest amplicon read depths from the first multiplex PCR amplification vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). In some embodiments, the highest and lowest primer pair concentrations associated with the second primer pair concentration profile vary by at least 2-fold (such as by at least any of 3-fold, 4-fold, S-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more).

In some embodiments, the method further comprises determining sequence read depths of amplicons from the second multiplex PCR amplification (e.g., by NGS). In some embodiments, the amplicon read depths from the second multiplex PCR amplification have a CV of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amplicon read depths from the second multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less). In some embodiments, the method further comprises determining the amount of primer dimer formation (such as by NGS) following the first multiplex PCR amplification for at least a subset of primers used in the first multiplex PCR amplification, and generating a filtered set of primer pairs based on the first set of primer pairs that excludes, for each primer dimer found to be present above a predetermined threshold, at least one of the primer pairs comprising a primer in the corresponding primer dimer.

In some embodiments, for the first or second multiplex PCR amplification, independently, each primer of at least a subset of primers used in the respective multiplex PCR amplification comprises at least one exonuclease-resistant internucleotide bond or at least one exonuclease-resistant nucleotide according to any of the embodiments described herein. In some embodiments, the at least 50 target loci comprises at least 100 (such as at least any of 100, 200, 300, 400, 500, 600, 700, 800, 900, 1000, or more) target loci. In some embodiments, the at least 50 target loci comprises at least 500 target loci. In some embodiments, the at least 50 target loci comprises at least 1000 target loci.

In some embodiments, a method of amplifying at least 50 target loci in a nucleic acid sample comprises (a) generating or obtaining a first set of primer pairs collectively comprising at least one primer pair for each of the at least 50 target loci; (b) performing a first multiplex PCR amplification on a first nucleic acid sample using the first set of primer pairs, wherein at least a subset of the first set of primer pairs are used in the first multiplex PCR amplification at concentrations according to a first primer pair concentration profile, thereby generating amplicons corresponding to the different primer pairs; (c) determining the amount of primer dimer formation (such as by NGS) following the first multiplex PCR amplification for at least a subset of primers used in the first multiplex PCR amplification, and generating a filtered set of primer pairs based on the first set of primer pairs that excludes, for each primer dimer found to be present above a predetermined threshold, at least one of the primer pairs comprising a primer in the corresponding primer dimer; (d) determining sequence read depths (such as by NGS) of amplicons from the first multiplex PCR amplification corresponding to the different primer pairs in the filtered set of primer pairs to generate a first amplification profile; (e) generating a second primer pair concentration profile based on the first amplification profile; and (f) performing a second multiplex PCR amplification on a second nucleic acid sample using the filtered set of primer pairs at concentrations according to the second primer pair concentration profile, thereby generating amplicons corresponding to the different primer pairs, wherein the concentration of each primer pair in the second primer pair concentration profile is selected based on the first amplification profile to produce amplicon read depths from the second multiplex PCR amplification that are closer to target read depths corresponding to the different primer pairs. In some embodiments, determining sequence read depths of amplicons is carried out by NGS.

In some embodiments, the first multiplex PCR amplification comprises a plurality of reactions, wherein for each reaction, each primer pair of the at least a subset of the first set of primer pairs is used, independently, in the reaction at one of a plurality of (such as at least 2, 3, 4, 5, 6, 7, 8, 9, 10, 11, 12, 13, 14, 15, or more) concentrations according to the first primer pair concentration profile. In some embodiments, the highest and lowest amplicon read depths from the first multiplex PCR amplification vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). In some embodiments, the highest and lowest primer pair concentrations associated with the second primer pair concentration profile vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more).

In some embodiments, the method further comprises determining sequence read depths of amplicons from the second multiplex PCR amplification (e.g., by NGS). In some embodiments, the amplicon read depths from the second multiplex PCR amplification have a CV of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amplicon read depths from the second multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less).

In some embodiments, for the first or second multiplex PCR amplification, independently, each primer of at least a subset of primers used in the respective multiplex PCR amplification comprises at least one exonuclease-resistant internucleotide bond or at least one exonuclease-resistant nucleotide according to any of the embodiments described herein. In some embodiments, the at least 50 target loci comprises at least 100 (such as at least any of 100, 200, 300, 400, 500, 600, 700, 800, 900, 1000, or more) target loci. In some embodiments, the at least 50 target loci comprises at least 500 target loci. In some embodiments, the at least 50 target loci comprises at least 1000 target loci.

In some embodiments, a method of amplifying at least 50 target loci in a nucleic acid sample comprises (a) generating or obtaining a first set of primer pairs collectively comprising at least one primer pair for each of the at least 50 target loci; (b) performing a first multiplex PCR amplification on a first nucleic acid sample using the first set of primer pairs, wherein the first multiplex PCR amplification comprises a plurality of reactions, wherein for each reaction, each primer pair of at least a subset of the first set of primer pairs is used, independently, in the reaction at one of a plurality of (such as at least 2, 3, 4, 5, 6, 7, 8, 9, 10, 11, 12, 13, 14, 15, or more) concentrations according to a first primer pair concentration profile, thereby generating amplicons corresponding to the different primer pairs; (c) determining sequence read depths of amplicons from the first multiplex PCR amplification to generate a first amplification profile; (d) generating a second primer pair concentration profile based on the first amplification profile; and (e) performing a second multiplex PCR amplification on a second nucleic acid sample using a second set of primer pairs selected from the first set of primer pairs, wherein at least a subset of the second set of primer pairs is used in the second multiplex PCR amplification at concentrations according to the second primer pair concentration profile, thereby generating amplicons corresponding to the different primer pairs, wherein the concentration of each primer pair in the second primer pair concentration profile is selected based on the first amplification profile produce amplicon read depths from the second multiplex PCR amplification that are closer to target read depths corresponding to the different primer pairs.

In some embodiments, the plurality of reactions collectively comprises each primer pair of the at least a subset of the first set of primer pairs at each concentration of the respective plurality of concentrations. In some embodiments, the plurality of concentrations collectively comprise concentrations ranging from between 100 pM and 10 pM (such as any of 100 pM, 150 pM, 200 pM, 250 pM, 300 pM, 350 pM, 400 pM, 450 pM, 500 pM, 550 pM, 600 pM, 650 pM, 700 pM, 750 pM, 800 pM, 850 pM, 900 pM, 950 pM, 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 220 nM, 240 nM, 260 nM, 280 nM, 300 nM, 320 nM, 340 nM, 360 nM, 380 nM, 400 nM, 420 nM, 440 nM, 460 nM, 480 nM, 500 nM, 520 nM, 540 nM, 560 nM, 580 nM, 600 nM, 620 nM, 640 nM, 660 nM, 680 nM, 700 nM, 720 nM, 740 nM, 760 nM, 780 nM, 800 nM, 820 nM, 840 nM, 860 nM, 880 nM, 900 nM, 920 nM, 940 nM, 960 nM, 980 nM, 1 μM, 2 μM, 3 μM, 4 μM, 5 μM, 6 μM, 7 μM, 8 μM, 9 μM, or 10 μM, including any ranges between any of these values). In some embodiments, the method further comprises using the first amplification profile to determine a relationship between primer pair concentration and sequence read depth for each primer pair of the at least a subset of the first set of primer pairs. In some embodiments, the relationship between primer pair concentration, or [P], and sequence read depth, or reads, is modeled as reads=b(2−a^{-[P]})²⁰. In some embodiments, the model is fit using a nonlinear least-squares fit. In some embodiments, the fit is performed in the log-log space.

In some embodiments, generating the second primer pair concentration profile based on the first amplification profile comprises using the relationship between primer pair concentration and sequence read depth for each primer pair of the at least a subset of the first set of primer pairs to determine the concentration for each primer pair of the at least a subset of the first set of primer pairs corresponding to a target sequence read depth. In some embodiments, determining sequence read depths of amplicons is carried out by NGS. In some embodiments, the highest and lowest amplicon read depths from the first multiplex PCR amplification vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). In some embodiments, the highest and lowest primer pair concentrations associated with the second primer pair concentration profile vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more).

In some embodiments, the method further comprises determining sequence read depths of amplicons from the second multiplex PCR amplification (e.g., by NGS). In some embodiments, the amplicon read depths from the second multiplex PCR amplification have a CV of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amplicon read depths from the second multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less).

In some embodiments, the method further comprises determining the amount of primer dimer formation (such as by NGS) following the first multiplex PCR amplification for at least a subset of primers used in the first multiplex PCR amplification, and generating a filtered set of primer pairs based on the first set of primer pairs that excludes, for each primer dimer found to be present above a predetermined threshold, at least one of the primer pairs comprising a primer in the corresponding primer dimer. In some embodiments, for the first or second multiplex PCR amplification, independently, each primer of at least a subset of primers used in the respective multiplex PCR amplification comprises at least one exonuclease-resistant internucleotide bond or at least one exonuclease-resistant nucleotide according to any of the embodiments described herein. In some embodiments, the at least 50 target loci comprises at least 100 (such as at least any of 100, 200, 300, 400, 500, 600, 700, 800, 900, 1000, or more) target loci. In some embodiments, the at least 50 target loci comprises at least 500 target loci. In some embodiments, the at least 50 target loci comprises at least 1000 target loci.

In some embodiments, a method of amplifying at least 50 target loci in a nucleic acid sample comprises (a) generating or obtaining a first set of primer pairs collectively comprising at least one primer pair for each of the at least 50 target loci; (b) performing a first multiplex PCR amplification on a first nucleic acid sample using the first set of primer pairs, wherein at least a subset of the first set of primer pairs are used in the first multiplex PCR amplification at concentrations according to a first primer pair concentration profile, thereby generating amplicons corresponding to the different primer pairs, (c) determining sequence read depths of amplicons from the first multiplex PCR amplification corresponding to the different primer pairs to generate a first amplification profile; (d) generating a second primer pair concentration profile based on the first amplification profile; and (e) performing a second multiplex PCR amplification on a second nucleic acid sample using a second set of primer pairs selected from the first set of primer pairs, wherein at least a subset of the second set of primer pairs is used in the second multiplex PCR amplification at concentrations according to the second primer pair concentration profile, thereby generating amplicons corresponding to the different primer pairs, wherein the concentration of each primer pair in the second primer pair concentration profile is selected based on the first amplification profile to produce amplicon read depths from the second multiplex PCR amplification that are closer to target read depths corresponding to the different primer pairs, and wherein for the first or second multiplex PCR amplification, independently, each primer of at least a subset of primers used in the respective multiplex PCR amplification comprises at least one exonuclease-resistant internucleotide bond or at least one exonuclease-resistant nucleotide according to any of the embodiments described herein.

In some embodiments, one or more primers of the at least a subset of primers comprises at least one exonuclease-resistant internucleotide bond at the 3′ end of the primer including, but not limited to, the five 3′ terminal nucleotides. In some embodiments, the exonuclease-resistant internucleotide bond is a phosphorothioate bond. In some embodiments, each primer of the one or more primers comprises at least 2 (such as 2, 3, 4, or more) phosphorothioate internucleotide bonds at the 3′ end of the primer. In some embodiments, each primer of the one or more primers comprises a phosphorothioate internucleotide bond between the nucleotide at the 0 position and the nucleotide at the −1 position and one or more phosphorothioate internucleotide bonds between any of (a) the nucleotide at the −1 position and the nucleotide at the −2 position; (b) the nucleotide at the −2 position and the nucleotide at the −3 position; and (c) the nucleotide at the −3 position and the nucleotide at the −4 position. In some embodiments, one or more primers of the at least a subset of primers comprises at least one exonuclease-resistant nucleotide at the 3′ end of the primer including, but not limited to, the five 3′ terminal nucleotides. In some embodiments, the exonuclease-resistant nucleotide is a locked nucleic acid (LNA) nucleotide, such as a nucleotide having a sugar moiety with a bridge connecting the 2′ oxygen and 4′ carbon. In some embodiments, each primer of the one or more primers comprises at least 2 (such as 2, 3, 4, or more) LNA nucleotides at the 3′ end of the primer. In some embodiments, each primer of the one or more primers comprises an LNA nucleotide at the 0 position and one or more LNA nucleotides at any of the −1, −2, and −3 positions. In some embodiments, the at least a subset of primers corresponds to at least 50% of the primers used in the respective multiplex PCR amplification.

In some embodiments, determining sequence read depths of amplicons is carried out by NGS. In some embodiments, the first multiplex PCR amplification comprises a plurality of reactions, wherein for each reaction, each primer pair of the at least a subset of the first set of primer pairs is used, independently, in the reaction at one of a plurality of (such as at least 2, 3, 4, 5, 6, 7, 8, 9, 10, 11, 12, 13, 14, 15, or more) concentrations according to the first primer pair concentration profile. In some embodiments, the highest and lowest amplicon read depths from the first multiplex PCR amplification vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). In some embodiments, the highest and lowest primer pair concentrations associated with the second primer pair concentration profile vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more).

In some embodiments, the method further comprises determining sequence read depths of amplicons from the second multiplex PCR amplification (e.g., by NGS). In some embodiments, the amplicon read depths from the second multiplex PCR amplification have a CV of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amplicon read depths from the second multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less).

In some embodiments, the method further comprises determining the amount of primer dimer formation (such as by NGS) following the first multiplex PCR amplification for at least a subset of primers used in the first multiplex PCR amplification, and generating a filtered set of primer pairs based on the first set of primer pairs that excludes, for each primer dimer found to be present above a predetermined threshold, at least one of the primer pairs comprising a primer in the corresponding primer dimer. In some embodiments, the at least 50 target loci comprise at least 100 (such as at least any of 100, 200, 300, 400, 500, 600, 700, 800, 900, 1000, or more) target loci. In some embodiments, the at least 50 target loci comprise at least 500 target loci. In some embodiments, the at least 50 target loci comprise at least 1000 target loci.

### Iterative Methods

The methods described above for multiplex PCR load balancing can be carried out iteratively, for example, to reach a desired set of amplicon target read depths.

Accordingly, in one aspect, a method of amplifying a plurality of target loci in a nucleic acid sample comprises (a) generating or obtaining a first set of primer pairs collectively comprising at least one primer pair for each of the plurality of target loci: (b) performing an N multiplex PCR amplification on an N nucleic acid sample using an N set of primer pairs selected from the first set of primer pairs, wherein at least a subset of the N set of primer pairs are used in the N multiplex PCR amplification at concentrations according to an N primer pair concentration profile, thereby generating amplicons corresponding to the different primer pairs, (c) determining sequence read depths of amplicons from the N multiplex PCR amplification corresponding to the different primer pairs to generate an N amplification profile; (d) generating an N+1 primer pair concentration profile based on the N amplification profile, wherein the concentrations of the primer pairs in the N+1 primer pair concentration profile are selected based on the N amplification profile to bring the amplicon read depths from an N+1 multiplex PCR amplification closer to target read depths corresponding to the different primer pairs used at concentrations according to the N+1 primer pair concentration profile; wherein steps (b) to (d) are carried out M−1 times for N=1 to M−1, followed by carrying out step (b) once for N=M, and wherein Mis an integer greater than I (such as greater than any of 2, 3, 4, 5, 6, 7, 8, 9, 10, 20, 30, 40, 50, or more). An M primer pair concentration profile generated according to the method is also referred to herein as a load-balanced primer pair concentration profile. In some embodiments, M is at least 5. In some embodiments, M is at least 10. In some embodiments, M is at least 50. In some embodiments, the plurality of target loci comprises at least 50 (such as at least any of 50, 100, 200, 300, 400, 500, 600, 700, 800, 900, 1000, or more) target loci. In some embodiments, the plurality of target loci comprises at least 100 target loci. In some embodiments, the plurality of target loci comprises at least 1000 target loci.

In some embodiments, an N multiplex PCR amplification (e.g., the 1 multiplex PCR amplification) comprises a plurality of reactions, wherein for each reaction, each primer pair of the at least a subset of the N set of primer pairs is used, independently, in the reaction at one of a plurality of (such as at least 2, 3, 4, 5, 6, 7, 8, 9, 10, 11, 12, 13, 14, 15, or more) concentrations according to the N primer pair concentration profile. In some embodiments, the plurality of reactions collectively comprises each primer pair of the at least a subset of the N set of primer pairs at each concentration of the respective plurality of concentrations. The plurality of concentrations for each primer pair in the N primer pair concentration profile need not be the same.

In some embodiments, the 1 primer pair concentration profile comprises, independently, each primer pair of the at least a subset of the 1 set of primer pairs at a plurality of (such as at least 2, 3, 4, 5, 6, 7, 8, 9, 10, 11, 12, 13, 14, 15, or more) concentrations. In some embodiments, the plurality of concentrations collectively comprise concentrations ranging from between 100 pM and 10 μM (such as any of 100 pM, 150 pM, 200 pM, 250 pM, 300 pM, 350 pM, 400 pM, 450 pM, 500 pM, 550 pM, 600 pM, 650 pM, 700 pM, 750 pM, 800 pM, 850 pM, 900 pM, 950 pM, 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 220 nM, 240 nM, 260 nM, 280 nM, 300 nM, 320 nM, 340 nM, 360 nM, 380 nM, 400 nM, 420 nM, 440 nM, 460 nM, 480 nM, 500 nM, 520 nM, 540 nM, 560 nM, 580 nM, 600 nM, 620 nM, 640 nM, 660 nM, 680 nM, 700 nM, 720 nM, 740 nM, 760 nM, 780 nM, 800 nM, 820 nM, 840 nM, 860 nM, 880 nM, 900 nM, 920 nM, 940 nM, 960 nM, 980 nM, 1 μM, 2 μM, 3 μM, 4 μM, 5 μM, 6 μM, 7 μM, 8 μM, 9 μM, or 10 μM, including any ranges between any of these values). In some embodiments, determining sequence read depths of amplicons from the 1 multiplex PCR amplification comprises determining sequence read depths of amplicons corresponding to each primer pair of the at least a subset of the 1 set of primer pairs at each of the respective plurality of concentrations to generate the 1 amplification profile.

In some embodiments, the method further comprises using the 1 amplification profile to determine a relationship between primer pair concentration and sequence read depth for each primer pair of the at least a subset of the 1 set of primer pairs. In some embodiments, the relationship between primer pair concentration, or [P], and sequence read depth, or reads, is modeled as reads=b(2−a^{-[P]})²⁰. In some embodiments, the model is fit using a nonlinear least-squares fit. In some embodiments, the fit is performed in the log-log space. In some embodiments, generating the 2 primer pair concentration profile based on the 1 amplification profile comprises using the relationship between primer pair concentration and sequence read depth for each primer pair of the at least a subset of the 1 set of primer pairs to determine a target concentration for each primer pair of the at least a subset of the 1 set of primer pairs corresponding to a target sequence read depth. Any suitable target sequence read depth can be selected, and this can vary depending on downstream applications of the multiplex PCR amplification. In some embodiments, the 2 primer pair concentration profile comprises, for each primer pair of the at least a subset of the 2 set of primer pairs, the respective primer pair at (a) the target concentration and (b) at least one (such as at least any of 2, 3, 4, 5, 6, or more) additional concentration based on the target concentration. In some embodiments, the at least one additional concentration based on the target concentration comprises (i) at least one (such as at least any of 2, 3, 4, 5, 6, or more) additional concentration that is lower than the target concentration or (i) at least one (such as at least any of 2, 3, 4, 5, 6, or more) additional concentration that is greater than the target concentration.

In some embodiments, according to any of the methods of amplifying a plurality of target loci described herein employing carrying out M multiplex PCR amplifications, generating an N+1 primer pair concentration profile based on the N amplification profile, where Nis an integer greater than 1, comprises using the N amplification profile to select an N+1 target concentration for each primer pair in the N+1 primer pair concentration profile. In some embodiments, the N+1 target concentration for a primer pair is selected based on one or more concentrations of the primer pair in the N amplification profile that correspond most closely to the target sequence read depth. For example, in some embodiments, the N+1 target concentration for a primer pair is selected to be (i) the concentration of the primer pair in the N amplification profile that corresponds most closely to the target sequence read depth, or (ii) a concentration based on two or more concentrations of the primer pair in the N amplification profile.

In some embodiments, N+1 target concentrations for multiple target sequences may be selected using high-throughput primer rebalancing performed in one or more iterative cycles. In some embodiments, high-throughput rebalancing is performed iteratively until desired target coverage has been reached or a balanced assay has been achieved. In some embodiments, high-throughput rebalancing comprises parallel testing of multiple primer concentrations. In some embodiments, high-throughput rebalancing comprises randomly assigning the primers and pooling into groups. In some embodiments, the pooling of primers may be performed manually by operators or using laboratory automation. In some embodiments, the pooling of primers may involve resuspension of lyophilized primers in a diluent. In some embodiments, the pooling of primers may involve starting material that has forward and reverse primers for the same target sequence in the same tube or well of a plate, or with the forward and reverse primer sequences separated. In some embodiments, the pooling of primers may involve using automated liquid handlers, semi-automated liquid handlers, or by hand. In some embodiments, the pooling of primers may involve exonuclease treatment before, during or after pooling. In some embodiments, the pooling of primers may involve dilution of primers to a custom concentration for a particular assay. In some embodiments, the pooling of primers may involve the use of a single, multiple, or no additional diluents, such as nuclease-free water, TE buffer (10 mM Tris-HCl, 1 mM EDTA), EB buffer (10 mM Tris-Cl, pH 8.5), or QCT buffer (0.05% Tween 20, 99.95% TE buffer). In some embodiments, high-throughput rebalancing comprises varying the primer concentrations for one group of primers while keeping the concentrations of the other groups of primers constant, and repeating the process for each group of primers so that each primer is tested at variable concentrations. In some embodiments, high-throughput rebalancing is performed through testing via multiple PCR reactions on a rebalancing plate. In some embodiments, each PCR reaction may comprise multiple steps such as enriching, tailing, and indexing PCRs performed on a variety of input materials. In some embodiments, the various input materials may include synthetic oligonucleotides, DNA extracted from specimen from biobanks or purchased from vendors. In some embodiments, the products of primer rebalancing are sequenced using NGS to create a titration curve of target sequence coverage relating primer concentration and target output. In some embodiments, the N+1 target concentration for each primer pair may be selected based on the titration curve.

In some embodiments, the N+1 target concentration may be selected using various interpolation and extrapolation techniques on the titration curve. For example, in some embodiments, the N+1 target concentration may be selected using nearest neighbor interpolation based on the data point that is closest to the target read depth. As another example, in some embodiments, the N+1 target concentration may be selected using linear interpolation based on the interpolation between two data points closest to the target read depth, where, if the target read depth is below the minimum depth, then the minimum concentration will be chosen as the N+1 target concentration, and if the target read depth is above the maximum depth, then the maximum concentration will be chosen as the N+1 target concentration. As another example, in some embodiments, the N+1 target concentration may be selected using a combination of linear interpolation and extrapolation, where, if the target read depth is below the minimum read depth or above the maximum read depth, two data points at the edge of the interpolation curve will be used to extrapolate to the target and the concentration of the extrapolated intersect will be chosen as the N+1 target concentration. In some embodiments, the maximum value for this extrapolation is set to 240 nM by default. In some embodiments, the maximum value for this extrapolation can be manually adjusted by operators. The N+1 target concentration for a primer pair in the N+1 primer pair concentration profile need not be different from the corresponding target concentration in the N primer pair concentration profile. In some embodiments, the N+1 primer pair concentration profile comprises, for each primer pair of the at least a subset of the N set of primer pairs, the respective primer pair at (a) the N+1 target concentration and (b) at least one (such as at least any of 2, 3, 4, 5, 6, or more) additional concentration based on the N+1 target concentration. In some embodiments, the at least one additional concentration based on the N+1 target concentration comprises (i) at least one (such as at least any of 2, 3, 4, 5, 6, or more) additional concentration that is lower than the N+1 target concentration or (i) at least one (such as at least any of 2, 3, 4, 5, 6, or more) additional concentration that is greater than the N+1 target concentration.

In some embodiments, NGS is used to determine sequence read depths of amplicons. For example, in some embodiments, the method further comprises steps of (i) processing a set of amplicons from a multiplex PCR amplification to be adapted for NGS, (ii) carrying out NGS on the processed amplicons, thereby generating sequence read data for the set of amplicons, and (iii) determining sequence read depths for the set of amplicons based on the sequence read data.

In some embodiments, the highest and lowest amplicon read depths from the 1 multiplex PCR amplification vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). For example, in some embodiments, the highest and lowest amplicon read depths from the 1 multiplex PCR amplification vary by at least 5-fold. In some embodiments, the highest and lowest amplicon read depths from the 1 multiplex PCR amplification vary by at least 10-fold. In some embodiments, the highest and lowest amplicon read depths from the 1 multiplex PCR amplification vary by at least 100-fold. In some embodiments, the highest and lowest amplicon read depths from the 1 multiplex PCR amplification vary by at least 1000-fold.

In some embodiments, the highest and lowest primer pair concentrations associated with the M primer pair concentration profile vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). For example, in some embodiments, the highest and lowest primer pair concentrations associated with the M primer pair concentration profile vary by at least 5-fold. In some embodiments, the highest and lowest primer pair concentrations associated with the M primer pair concentration profile vary by at least 10-fold. In some embodiments, the highest and lowest primer pair concentrations associated with the M primer pair concentration profile vary by at least 100-fold. In some embodiments, the highest and lowest primer pair concentrations associated with the M primer pair concentration profile vary by at least 1000-fold.

In some embodiments, the method further comprises determining sequence read depths of amplicons from the M multiplex PCR amplification, wherein the amplicon read depths from the M multiplex PCR amplification have a coefficient of variance (CV) of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). For example, in some embodiments, the amplicon read depths from the M multiplex PCR amplification corresponding to the different primer pairs have a CV of less than 10%. In some embodiments, the amplicon read depths from the M multiplex PCR amplification corresponding to the different primer pairs have a CV of less than 5%. In some embodiments, the amplicon read depths from the M multiplex PCR amplification corresponding to the different primer pairs have a CV of less than 2%. In some embodiments, the amplicon read depths from the M multiplex PCR amplification corresponding to the different primer pairs have a CV of less than 1%.

In some embodiments, the method further comprises determining sequence read depths of amplicons from the M multiplex PCR amplification, wherein the amplicon read depths from the M multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less). For example, in some embodiments, the amplicon read depths from the M multiplex PCR amplification corresponding to the different primer pairs have a 90^{th} percentile over 10^{th} percentile of less than 1.4. In some embodiments, the amplicon read depths from the M multiplex PCR amplification corresponding to the different primer pairs have a 90^{th} percentile over 10^{th} percentile of less than 1.25. In some embodiments, the amplicon read depths from the M multiplex PCR amplification corresponding to the different primer pairs have a 90^{th} percentile over 10^{th} percentile of less than 1.1. In some embodiments, the amplicon read depths from the M multiplex PCR amplification corresponding to the different primer pairs have a 90^{th} percentile over 10^{th} percentile of less than 1.05.

In some embodiments, the method further comprises determining the amount of primer dimer formation (such as by NGS) following an X multiplex PCR amplification (e.g., the 1 multiplex PCR amplification) for at least a subset of primers used in the X multiplex PCR amplification, where X is any of 1 to M−1, and generating a filtered set of primer pairs based on the X set of primer pairs that excludes, for each primer dimer found to be present above a predetermined threshold, at least one of the primer pairs comprising a primer in the corresponding primer dimer. In some embodiments, the filtered set of primer pairs is used in one or more subsequent multiplex PCR amplifications (e.g., each of the N multiplex PCR amplifications for N=X+1 to M). In some embodiments, the predetermined threshold is 1/N.

In some embodiments, the method further comprises determining the amount of primer dimer formation (such as by NGS) following the M multiplex PCR amplification for at least a subset of primers used in the M multiplex PCR amplification, and generating a filtered set of primer pairs based on the M set of primer pairs that excludes, for each primer dimer found to be present above a predetermined threshold, at least one of the primer pairs comprising a primer in the corresponding primer dimer. In some embodiments, the predetermined threshold is 1/N, where N is the number of primer pairs.

In some embodiments, for one or more of the N multiplex PCR amplifications, independently, each primer of at least a subset of primers used in the respective multiplex PCR amplification comprises at least one exonuclease-resistant internucleotide bond or at least one exonuclease-resistant nucleotide according to any of the embodiments described herein. In some embodiments, one or more primers of the at least a subset of primers comprises at least one exonuclease-resistant internucleotide bond at the 3′ end of the primer including, but not limited to, the five 3′ terminal nucleotides. In some embodiments, the exonuclease-resistant internucleotide bond is a phosphorothioate bond. In some embodiments, each primer of the one or more primers comprises at least 2 (such as 2, 3, 4, or more) phosphorothioate internucleotide bonds at the 3′ end of the primer. In some embodiments, each primer of the one or more primers comprises a phosphorothioate internucleotide bond between the nucleotide at the 0 position and the nucleotide at the −1 position and one or more phosphorothioate internucleotide bonds between any of (a) the nucleotide at the −1 position and the nucleotide at the −2 position; (b) the nucleotide at the −2 position and the nucleotide at the −3 position; and (c) the nucleotide at the −3 position and the nucleotide at the −4 position. In some embodiments, one or more primers of the at least a subset of primers comprises at least one exonuclease-resistant nucleotide at the 3′ end of the primer including, but not limited to, the five 3′ terminal nucleotides. In some embodiments, the exonuclease-resistant nucleotide is a locked nucleic acid (LNA) nucleotide, such as a nucleotide having a sugar moiety with a bridge connecting the 2′ oxygen and 4′ carbon. In some embodiments, each primer of the one or more primers comprises at least 2 (such as 2, 3, 4, or more) LNA nucleotides at the 3′ end of the primer. In some embodiments, each primer of the one or more primers comprises an LNA nucleotide at the 0 position and one or more LNA nucleotides at any of the −1, −2, and −3 positions. In some embodiments, the at least a subset of primers corresponds to at least 50% of the primers used in the respective multiplex PCR amplification.

In some embodiments, a method of amplifying at least 50 target loci in a nucleic acid sample comprises (a) generating or obtaining a first set of primer pairs collectively comprising at least one primer pair for each of the at least 50 target loci; (b) performing an N multiplex PCR amplification on an N nucleic acid sample using an N set of primer pairs selected from the first set of primer pairs, wherein at least a subset of the N set of primer pairs are used in the N multiplex PCR amplification at concentrations according to an N primer pair concentration profile, thereby generating amplicons corresponding to the different primer pairs; (c) determining sequence read depths of amplicons from the N multiplex PCR amplification corresponding to the different primer pairs to generate an N amplification profile; (d) generating an N+1 primer pair concentration profile based on the N amplification profile, wherein the concentrations of the primer pairs in the N+1 primer pair concentration profile are selected based on the N amplification profile to bring the amplicon read depths from an N+1 multiplex PCR amplification closer to target read depths corresponding to the different primer pairs used at concentrations according to the N+1 primer pair concentration profile, wherein steps (b) to (d) are carried out M−1 times for N=1 to M−1, followed by carrying out step (b) once for N−M, and wherein Mis an integer greater than 1 (such as greater than any of 2, 3, 4, 5, 6, 7, 8, 9, 10, 20, 30, 40, 50, or more). In some embodiments, determining sequence read depths of amplicons is carried out by NGS.

In some embodiments, an N multiplex PCR amplification (e.g., the 1 multiplex PCR amplification) comprises a plurality of reactions, wherein for each reaction, each primer pair of the at least a subset of the N set of primer pairs is used, independently, in the reaction at one of a plurality of (such as at least 2, 3, 4, 5, 6, 7, 8, 9, 10, 11, 12, 13, 14, 15, or more) concentrations according to the N primer pair concentration profile, and wherein the plurality of reactions collectively comprise each primer pair of the at least a subset of the N set of primer pairs at each concentration of the respective plurality of concentrations. In some embodiments, the highest and lowest amplicon read depths from the 1 multiplex PCR amplification vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). In some embodiments, the highest and lowest primer pair concentrations associated with the M primer pair concentration profile vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more).

In some embodiments, the method further comprises determining sequence read depths of amplicons from the M multiplex PCR amplification (e.g., by NGS). In some embodiments, the amplicon read depths from the M multiplex PCR amplification have a CV of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amplicon read depths from the M multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less).

In some embodiments, the method further comprises determining the amount of primer dimer formation (such as by NGS) following an X multiplex PCR amplification (e.g., the I multiplex PCR amplification) for at least a subset of primers used in the X multiplex PCR amplification, where X is any of 1 to M−1, and generating a filtered set of primer pairs based on the X set of primer pairs that excludes, for each primer dimer found to be present above a predetermined threshold, at least one of the primer pairs comprising a primer in the corresponding primer dimer. In some embodiments, the filtered set of primer pairs is used in one or more subsequent multiplex PCR amplifications. In some embodiments, for one or more of the N multiplex PCR amplifications, independently, each primer of at least a subset of primers used in the respective multiplex PCR amplification comprises at least one exonuclease-resistant internucleotide bond or at least one exonuclease-resistant nucleotide according to any of the embodiments described herein. In some embodiments, the at least 50 target loci comprises at least 100 (such as at least any of 100, 200, 300, 400, 500, 600, 700, 800, 900, 1000, or more) target loci. In some embodiments, the at least 50 target loci comprises at least 500 target loci. In some embodiments, the at least 50 target loci comprises at least 1000 target loci.

In some embodiments a method of amplifying at least 50 target loci in a nucleic acid sample comprises (a) generating or obtaining a first set of primer pairs collectively comprising at least one primer pair for each of the at least 50 target loci; (b) performing an N multiplex PCR amplification on an N nucleic acid sample using an N set of primer pairs selected from the first set of primer pairs, wherein at least a subset of the N set of primer pairs are used in the N multiplex PCR amplification at concentrations according to an N primer pair concentration profile, thereby generating amplicons corresponding to the different primer pairs; (c) determining, by NGS, sequence read depths of amplicons from the N multiplex PCR amplification corresponding to the different primer pairs to generate an N amplification profile; (d) generating an N+1 primer pair concentration profile based on the N amplification profile, wherein the concentrations of the primer pairs in the N+1 primer pair concentration profile are selected based on the N amplification profile to bring the amplicon read depths from an N+1 multiplex PCR amplification closer to target read depths corresponding to the different primer pairs used at concentrations according to the N+1 primer pair concentration profile; wherein steps (b) to (d) are carried out M−1 times for N=1 to M−1, followed by carrying out step (b) once for N=M, and wherein Mis an integer greater than 1 (such as greater than any of 2, 3, 4, 5, 6, 7, 8, 9, 10, 20, 30, 40, 50, or more).

In some embodiments, the method further comprises (i) processing a set of amplicons from a multiplex PCR amplification to be adapted for NGS, (ii) carrying out NGS on the processed amplicons, thereby generating sequence read data for the set of amplicons, and (iii) determining sequence read depths for the set of amplicons based on the sequence read data.

In some embodiments, an N multiplex PCR amplification (e.g., the 1 multiplex PCR amplification) comprises a plurality of reactions, wherein for each reaction, each primer pair of the at least a subset of the N set of primer pairs is used, independently, in the reaction at one of a plurality of (such as at least 2, 3, 4, 5, 6, 7, 8, 9, 10, 11, 12, 13, 14, 15, or more) concentrations according to the N primer pair concentration profile, and wherein the plurality of reactions collectively comprise each primer pair of the at least a subset of the N set of primer pairs at each concentration of the respective plurality of concentrations. In some embodiments, the highest and lowest amplicon read depths from the 1 multiplex PCR amplification vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). In some embodiments, the highest and lowest primer pair concentrations associated with the M primer pair concentration profile vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more).

In some embodiments, the method further comprises determining sequence read depths of amplicons from the M multiplex PCR amplification (e.g., by NGS). In some embodiments, the amplicon read depths from the M multiplex PCR amplification have a CV of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amplicon read depths from the M multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less). In some embodiments, the method further comprises determining the amount of primer dimer formation (such as by NGS) following an X multiplex PCR amplification (e.g., the 1 multiplex PCR amplification) for at least a subset of primers used in the X multiplex PCR amplification, where X is any of 1 to M−1, and generating a filtered set of primer pairs based on the X set of primer pairs that excludes, for each primer dimer found to be present above a predetermined threshold, at least one of the primer pairs comprising a primer in the corresponding primer dimer. In some embodiments, the filtered set of primer pairs is used in one or more subsequent multiplex PCR amplifications. In some embodiments, for one or more of the N multiplex PCR amplifications, independently, each primer of at least a subset of primers used in the respective multiplex PCR amplification comprises at least one exonuclease-resistant internucleotide bond or at least one exonuclease-resistant nucleotide according to any of the embodiments described herein. In some embodiments, the at least 50 target loci comprises at least 100 (such as at least any of 100, 200, 300, 400, 500, 600, 700, 800, 900, 1000, or more) target loci. In some embodiments, the at least 50 target loci comprises at least 500 target loci. In some embodiments, the at least 50 target loci comprises at least 1000 target loci.

In some embodiments, a method of amplifying at least 50 target loci in a nucleic acid sample comprises (a) generating or obtaining a first set of primer pairs collectively comprising at least one primer pair for each of the at least 50 target loci; (b) performing an N multiplex PCR amplification on an N nucleic acid sample using an N set of primer pairs selected from the first set of primer pairs and excluding any primer pairs in a set of blacklisted primer pairs, wherein at least a subset of the N set of primer pairs are used in the N multiplex PCR amplification at concentrations according to an N primer pair concentration profile, thereby generating amplicons corresponding to the different primer pairs; (c) optionally determining the amount of primer dimer formation (such as by NGS) following the N multiplex PCR amplification for at least a subset of primers used in the N multiplex PCR amplification, and, for each primer dimer found to be present above a predetermined threshold, adding at least one of the primer pairs comprising a primer in the corresponding primer dimer to the set of blacklisted primer pairs; (d) determining sequence read depths of amplicons from the N multiplex PCR amplification corresponding to a filtered N set of primer pairs excluding any primer pairs in the set of blacklisted primer pairs to generate an N amplification profile; and (e) generating an N+1 primer pair concentration profile based on the N amplification profile, wherein the concentrations of the primer pairs in the N+1 primer pair concentration profile are selected based on the N amplification profile to bring the amplicon read depths from an N+1 multiplex PCR amplification closer to target read depths corresponding to the filtered N set of primer pairs used at concentrations according to the N+1 primer pair concentration profile; wherein steps (b) to (e) are carried out M−1 times for N=1 to M−1, followed by carrying out step (b) once for N=M, wherein Mis an integer greater than 1 (such as greater than any of 2, 3, 4, 5, 6, 7, 8, 9, 10, 20, 30, 40, 50, or more). In some embodiments, determining sequence read depths of amplicons is carried out by NGS.

In some embodiments, an N multiplex PCR amplification (e.g., the 1 multiplex PCR amplification) comprises a plurality of reactions, wherein for each reaction, each primer pair of the at least a subset of the N set of primer pairs is used, independently, in the reaction at one of a plurality of (such as at least 2, 3, 4, 5, 6, 7, 8, 9, 10, 11, 12, 13, 14, 15, or more) concentrations according to the N primer pair concentration profile, and wherein the plurality of reactions collectively comprise each primer pair of the at least a subset of the N set of primer pairs at each concentration of the respective plurality of concentrations. In some embodiments, the highest and lowest amplicon read depths from the 1 multiplex PCR amplification vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). In some embodiments, the highest and lowest primer pair concentrations associated with the M primer pair concentration profile vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more).

In some embodiments, the method further comprises determining sequence read depths of amplicons from the M multiplex PCR amplification (e.g., by NGS). In some embodiments, the amplicon read depths from the M multiplex PCR amplification have a CV of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amplicon read depths from the M multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less). In some embodiments, for one or more of the N multiplex PCR amplifications, independently, each primer of at least a subset of primers used in the respective multiplex PCR amplification comprises at least one exonuclease-resistant internucleotide bond or at least one exonuclease-resistant nucleotide according to any of the embodiments described herein. In some embodiments, the at least 50 target loci comprises at least 100 (such as at least any of 100, 200, 300, 400, 500, 600, 700, 800, 900, 1000, or more) target loci. In some embodiments, the at least 50 target loci comprises at least 500 target loci. In some embodiments, the at least 50 target loci comprises at least 1000 target loci.

In some embodiments, a method of amplifying at least 50 target loci in a nucleic acid sample comprises (a) generating or obtaining a first set of primer pairs collectively comprising at least one primer pair for each of the at least 50 target loci; (b) performing an N multiplex PCR amplification on an N nucleic acid sample using an N set of primer pairs selected from the first set of primer pairs, wherein at least a subset of the N set of primer pairs are used in the N multiplex PCR amplification at concentrations according to an N primer pair concentration profile, thereby generating amplicons corresponding to the different primer pairs; (c) determining sequence read depths of amplicons from the N multiplex PCR amplification corresponding to the different primer pairs to generate an N amplification profile; and (d) generating an N+1 primer pair concentration profile based on the N amplification profile, wherein the concentrations of the primer pairs in the N+1 primer pair concentration profile are selected based on the N amplification profile to bring the amplicon read depths from an N+1 multiplex PCR amplification closer to target read depths corresponding to the filtered N set of primer pairs used at concentrations according to the N+1 primer pair concentration profile; wherein steps (b) to (d) are carried out M−1 times for N=1 to M−1, followed by carrying out step (b) once for N=M, wherein Mis an integer greater than 1 (such as greater than any of 2, 3, 4, 5, 6, 7, 8, 9, 10, 20, 30, 40, 50, or more), and wherein the amount of primer dimer formation is determined (such as by NGS) following the 1 multiplex PCR amplification for at least a subset of primers used in the 1 multiplex PCR amplification, and the 1 set of primer pairs is selected from the first set of primer pairs and to exclude, for each primer dimer found to be present above a predetermined threshold, at least one of the primer pairs comprising a primer in the corresponding primer dimer. In some embodiments, determining sequence read depths of amplicons is carried out by NGS.

In some embodiments, an N multiplex PCR amplification (e.g., the 1 multiplex PCR amplification) comprises a plurality of reactions, wherein for each reaction, each primer pair of the at least a subset of the N set of primer pairs is used, independently, in the reaction at one of a plurality of (such as at least 2, 3, 4, 5, 6, 7, 8, 9, 10, 11, 12, 13, 14, 15, or more) concentrations according to the N primer pair concentration profile, and wherein the plurality of reactions collectively comprise each primer pair of the at least a subset of the N set of primer pairs at each concentration of the respective plurality of concentrations. In some embodiments, the highest and lowest amplicon read depths from the 1 multiplex PCR amplification vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). In some embodiments, the highest and lowest primer pair concentrations associated with the M primer pair concentration profile vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more).

In some embodiments, the method further comprises determining sequence read depths of amplicons from the M multiplex PCR amplification (e.g., by NGS). In some embodiments, the amplicon read depths from the M multiplex PCR amplification have a CV of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amplicon read depths from the M multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less). In some embodiments, for one or more of the N multiplex PCR amplifications, independently, each primer of at least a subset of primers used in the respective multiplex PCR amplification comprises at least one exonuclease-resistant internucleotide bond or at least one exonuclease-resistant nucleotide according to any of the embodiments described herein. In some embodiments, the at least 50 target loci comprises at least 100 (such as at least any of 100, 200, 300, 400, 500, 600, 700, 800, 900, 1000, or more) target loci. In some embodiments, the at least 50 target loci comprises at least 500 target loci. In some embodiments, the at least 50 target loci comprises at least 1000 target loci.

In some embodiments, a method of amplifying at least 50 target loci in a nucleic acid sample comprises (a) generating or obtaining a first set of primer pairs collectively comprising at least one primer pair for each of the at least 50 target loci; (b) performing an N multiplex PCR amplification on an N nucleic acid sample using an N set of primer pairs selected from the first set of primer pairs, wherein the N multiplex PCR amplification comprises a plurality of reactions, wherein for each reaction, each primer pair of at least a subset of the N set of primer pairs is used, independently, in the reaction at one of a plurality of (such as at least 2, 3, 4, 5, 6, 7, 8, 9, 10, 11, 12, 13, 14, 15, or more) concentrations according to an N primer pair concentration profile, thereby generating amplicons corresponding to the different primer pairs; (c) determining sequence read depths of amplicons from the N multiplex PCR amplification corresponding to the different primer pairs to generate an N amplification profile; (d) generating an N+1 primer pair concentration profile based on the N amplification profile, wherein the concentrations of the primer pairs in the N+1 primer pair concentration profile are selected based on the N amplification profile to bring the amplicon read depths from an N+1 multiplex PCR amplification closer to target read depths corresponding to the different primer pairs used at concentrations according to the N+1 primer pair concentration profile; wherein steps (b) to (d) are carried out M−1 times for N=1 to M−1, followed by carrying out step (b) once for N−M, and wherein Mis an integer greater than 1 (such as greater than any of 2, 3, 4, 5, 6, 7, 8, 9, 10, 20, 30, 40, 50, or more).

In some embodiments, the plurality of reactions collectively comprise each primer pair of the at least a subset of the N set of primer pairs at each concentration of the respective plurality of concentrations. In some embodiments, the plurality of concentrations collectively comprise concentrations ranging from between 100 pM and 10 μM (such as any of 100 pM, 150 pM, 200 pM, 250 pM, 300 pM, 350 pM, 400 pM, 450 pM, 500 pM, 550 pM, 600 pM, 650 pM, 700 pM, 750 pM, 800 pM, 850 pM, 900 pM, 950 pM, 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 220 nM, 240 nM, 260 nM, 280 nM, 300 nM, 320 nM, 340 nM, 360 nM, 380 nM, 400 nM, 420 nM, 440 nM, 460 nM, 480 nM, 500 nM, 520 nM, 540 nM, 560 nM, 580 nM, 600 nM, 620 nM, 640 nM, 660 nM, 680 nM, 700 nM, 720 nM, 740 nM, 760 nM, 780 nM, 800 nM, 820 nM, 840 nM, 860 nM, 880 nM, 900 nM, 920 nM, 940 nM, 960 nM, 980 nM, 1 μM, 2 μM, 3 μM, 4 μM, 5 μM, 6 μM, 7 μM, 8 μM, 9 μM, or 10 μM, including any ranges between any of these values).

In some embodiments, determining sequence read depths of amplicons from the N multiplex PCR amplification comprises determining sequence read depths of amplicons corresponding to each primer pair of the at least a subset of the N set of primer pairs at each of the respective plurality of concentrations to generate the N amplification profile. In some embodiments, the method further comprises using the N amplification profile to determine a relationship between primer pair concentration and sequence read depth for each primer pair of the at least a subset of the N set of primer pairs. In some embodiments, the relationship between primer pair concentration, or [P], and sequence read depth, or reads, is modeled as reads=b(2−a^{-[P]})²⁰. In some embodiments, the model is fit using a nonlinear least-squares fit. In some embodiments, the fit is performed in the log-log space. In some embodiments, generating the N+1 primer pair concentration profile based on the N amplification profile comprises using the relationship between primer pair concentration and sequence read depth for each primer pair of the at least a subset of the N set of primer pairs.

In some embodiments, determining sequence read depths of amplicons is carried out by NGS. In some embodiments, the highest and lowest amplicon read depths from the 1 multiplex PCR amplification vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). In some embodiments, the highest and lowest primer pair concentrations associated with the M primer pair concentration profile vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). In some embodiments, the method further comprises determining sequence read depths of amplicons from the M multiplex PCR amplification (e.g., by NGS). In some embodiments, the amplicon read depths from the M multiplex PCR amplification have a CV of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amplicon read depths from the M multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less). In some embodiments, the method further comprises determining the amount of primer dimer formation (such as by NGS) following an X multiplex PCR amplification (e.g., the 1 multiplex PCR amplification) for at least a subset of primers used in the X multiplex PCR amplification, where X is any of 1 to M−1, and generating a filtered set of primer pairs based on the X set of primer pairs that excludes, for each primer dimer found to be present above a predetermined threshold, at least one of the primer pairs comprising a primer in the corresponding primer dimer.

In some embodiments, the filtered set of primer pairs is used in one or more subsequent multiplex PCR amplifications. In some embodiments, for one or more of the N multiplex PCR amplifications, independently, each primer of at least a subset of primers used in the respective multiplex PCR amplification comprises at least one exonuclease-resistant internucleotide bond or at least one exonuclease-resistant nucleotide according to any of the embodiments described herein. In some embodiments, the at least 50 target loci comprises at least 100 (such as at least any of 100, 200, 300, 400, 500, 600, 700, 800, 900, 1000, or more) target loci. In some embodiments, the at least 50 target loci comprises at least 500 target loci. In some embodiments, the at least 50 target loci comprises at least 1000 target loci.

In some embodiments, a method of amplifying at least 50 target loci in a nucleic acid sample comprising (a) generating or obtaining a first set of primer pairs collectively comprising at least one primer pair for each of the at least 50 target loci; (b) performing an N multiplex PCR amplification on an N nucleic acid sample using an N set of primer pairs selected from the first set of primer pairs, wherein the N multiplex PCR amplification comprises a plurality of reactions, wherein for each reaction, each primer pair of at least a subset of the N set of primer pairs is used, independently, in the reaction at one of a plurality of (such as at least 2, 3, 4, 5, 6, 7, 8, 9, 10, 11, 12, 13, 14, 15, or more) concentrations according to an N primer pair concentration profile, thereby generating amplicons corresponding to the different primer pairs; (c) determining sequence read depths of amplicons from the N multiplex PCR amplification corresponding to the different primer pairs to generate an N amplification profile; (d) generating an N+1 primer pair concentration profile based on the N amplification profile, wherein the concentrations of the primer pairs in the N+1 primer pair concentration profile are selected based on the N amplification profile to bring the amplicon read depths closer to target read depths from an N+1 multiplex PCR amplification corresponding to the different primer pairs used at concentrations according to the N+1 primer pair concentration profile; wherein steps (b) to (d) are carried out M−1 times for N=1 to M−1, followed by carrying out step (b) once for N=M, wherein M is an integer greater than 1 (such as greater than any of 2, 3, 4, 5, 6, 7, 8, 9, 10, 20, 30, 40, 50, or more), and wherein the 1 amplification profile is used to determine a relationship between primer pair concentration and sequence read depth for each primer pair of the at least a subset of the 1 set of primer pairs, and generating the 2 primer pair concentration profile based on the 1 amplification profile comprises using the relationship between primer pair concentration and sequence read depth for each primer pair of the at least a subset of the 1 set of primer pairs.

In some embodiments, generating the 2 primer pair concentration profile based on the 1 amplification profile comprises using the relationship between primer pair concentration and sequence read depth for each primer pair of the at least a subset of the 1 set of primer pairs to determine a target concentration for each primer pair of the at least a subset of the 1 set of primer pairs corresponding to a target sequence read depth. In some embodiments, the 2 primer pair concentration profile comprises, for each primer pair of the at least a subset of the 2 set of primer pairs, the respective primer pair at (a) the target concentration and (b) at least one (such as at least any of 2, 3, 4, 5, 6, or more) additional concentration based on the target concentration. In some embodiments, the at least one additional concentration based on the target concentration comprises (i) at least one (such as at least any of 2, 3, 4, 5, 6, or more) additional concentration that is lower than the target concentration or (i) at least one (such as at least any of 2, 3, 4, 5, 6, or more) additional concentration that is greater than the target concentration.

In some embodiments, generating an N+1 primer pair concentration profile based on the N amplification profile, where Nis an integer greater than 1, comprises using the N amplification profile to select an N+1 target concentration for each primer pair in the N+1 primer pair concentration profile. In some embodiments, the N+1 target concentration for a primer pair is selected based on one or more concentrations of the primer pair in the N amplification profile that correspond most closely to the target sequence read depth. For example, in some embodiments, the N+1 target concentration for a primer pair is selected to be (i) the concentration of the primer pair in the N amplification profile that corresponds most closely to the target sequence read depth, or (ii) a concentration based on two or more concentrations of the primer pair in the N amplification profile.

In some embodiments, the plurality of reactions collectively comprise each primer pair of the at least a subset of the N set of primer pairs at each concentration of the respective plurality of concentrations. In some embodiments, the plurality of concentrations collectively comprise concentrations ranging from between 100 pM and 10 pM (such as any of 100 pM, 150 pM, 200 pM, 250 pM, 300 pM, 350 pM, 400 pM, 450 pM, 500 pM, 550 pM, 600 pM, 650 pM, 700 pM, 750 pM, 800 pM, 850 pM, 900 pM, 950 pM, 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 220 nM, 240 nM, 260 nM, 280 nM, 300 nM, 320 nM, 340 nM, 360 nM, 380 nM, 400 nM, 420 nM, 440 nM, 460 nM, 480 nM, 500 nM, 520 nM, 540 nM, 560 nM, 580 nM, 600 nM, 620 nM, 640 nM, 660 nM, 680 nM, 700 nM, 720 nM, 740 nM, 760 nM, 780 nM, 800 nM, 820 nM, 840 nM, 860 nM, 880 nM, 900 nM, 920 nM, 940 nM, 960 nM, 980 nM, 1 μM, 2 μM, 3 μM, 4 μM, 5 μM, 6 μM, 7 μM, 8 μM, 9 μM, or 10 μM, including any ranges between any of these values).

In some embodiments, determining sequence read depths of amplicons from the N multiplex PCR amplification comprises determining sequence read depths of amplicons corresponding to each primer pair of the at least a subset of the N set of primer pairs at each of the respective plurality of concentrations to generate the N amplification profile. In some embodiments, the relationship between primer pair concentration, or [P], and sequence read depth, or reads, is modeled as reads=b(2−a^{-[P]})²⁰. In some embodiments, the model is fit using a nonlinear least-squares fit. In some embodiments, the fit is performed in the log-log space. In some embodiments, determining sequence read depths of amplicons is carried out by NGS. In some embodiments, the highest and lowest amplicon read depths from the 1 multiplex PCR amplification vary by at least 2-fold (such as by at least any of 3-fold, 4-fold, S-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). In some embodiments, the highest and lowest primer pair concentrations associated with the M primer pair concentration profile vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more).

In some embodiments, the method further comprises determining sequence read depths of amplicons from the M multiplex PCR amplification (e.g., by NGS). In some embodiments, the amplicon read depths from the M multiplex PCR amplification have a CV of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amplicon read depths from the M multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less). In some embodiments, the method further comprises determining the amount of primer dimer formation (such as by NGS) following an X multiplex PCR amplification (e.g., the 1 multiplex PCR amplification) for at least a subset of primers used in the X multiplex PCR amplification, where X is any of 1 to M−1, and generating a filtered set of primer pairs based on the A set of primer pairs that excludes, for each primer dimer found to be present above a predetermined threshold, at least one of the primer pairs comprising a primer in the corresponding primer dimer. In some embodiments, the filtered set of primer pairs is used in one or more subsequent multiplex PCR amplifications.

In some embodiments, for one or more of the N multiplex PCR amplifications, independently, each primer of at least a subset of primers used in the respective multiplex PCR amplification comprises at least one exonuclease-resistant internucleotide bond or at least one exonuclease-resistant nucleotide according to any of the embodiments described herein. In some embodiments, the at least 50 target loci comprises at least 100 (such as at least any of 100, 200, 300, 400, 500, 600, 700, 800, 900, 1000, or more) target loci. In some embodiments, the at least 50 target loci comprises at least 500 target loci. In some embodiments, the at least 50 target loci comprises at least 1000 target loci.

In some embodiments, a method of amplifying at least 50 target loci in a nucleic acid sample comprises (a) generating or obtaining a first set of primer pairs collectively comprising at least one primer pair for each of the at least 50 target loci; (b) performing an N multiplex PCR amplification on an N nucleic acid sample using an N set of primer pairs selected from the first set of primer pairs, wherein at least a subset of the N set of primer pairs are used in the N multiplex PCR amplification at concentrations according to an N primer pair concentration profile, thereby generating amplicons corresponding to the different primer pairs; (c) determining sequence read depths of amplicons from the N multiplex PCR amplification corresponding to the different primer pairs to generate an N amplification profile; (d) generating an N+1 primer pair concentration profile based on the N amplification profile, wherein the concentrations of the primer pairs in the N+1 primer pair concentration profile are selected based on the N amplification profile to bring the amplicon read depths from an N+1 multiplex PCR amplification closer to target read depths corresponding to the different primer pairs used at concentrations according to the N+1 primer pair concentration profile; wherein steps (b) to (d) are carried out M−1 times for N=1 to M−1, followed by carrying out step (b) once for N−M, and wherein Mis an integer greater than 1 (such as greater than any of 2, 3, 4, 5, 6, 7, 8, 9, 10, 20, 30, 40, 50, or more), and wherein for one or more of the N multiplex PCR amplifications, independently, each primer of at least a subset of primers used in the respective multiplex PCR amplification comprises at least one exonuclease-resistant internucleotide bond or at least one exonuclease-resistant nucleotide.

In some embodiments, one or more primers of the at least a subset of primers comprises at least one exonuclease-resistant internucleotide bond at the 3′ end of the primer including, but not limited to, the five 3′ terminal nucleotides. In some embodiments, the exonuclease-resistant internucleotide bond is a phosphorothioate bond. In some embodiments, each primer of the one or more primers comprises at least 2 (such as 2, 3, 4, or more) phosphorothioate internucleotide bonds at the 3′ end of the primer. In some embodiments, each primer of the one or more primers comprises a phosphorothioate internucleotide bond between the nucleotide at the 0 position and the nucleotide at the −1 position and one or more phosphorothioate internucleotide bonds between any of (a) the nucleotide at the −1 position and the nucleotide at the −2 position; (b) the nucleotide at the −2 position and the nucleotide at the −3 position; and (c) the nucleotide at the −3 position and the nucleotide at the −4 position. In some embodiments, one or more primers of the at least a subset of primers comprises at least one exonuclease-resistant nucleotide at the 3′ end of the primer including, but not limited to, the five 3′ terminal nucleotides. In some embodiments, the exonuclease-resistant nucleotide is a locked nucleic acid (LNA) nucleotide, such as a nucleotide having a sugar moiety with a bridge connecting the 2′ oxygen and 4′ carbon. In some embodiments, each primer of the one or more primers comprises at least 2 (such as 2, 3, 4, or more) LNA nucleotides at the 3′ end of the primer. In some embodiments, each primer of the one or more primers comprises an LNA nucleotide at the 0 position and one or more LNA nucleotides at any of the −1, −2, and −3 positions. In some embodiments, the at least a subset of primers corresponds to at least 50% of the primers used in the respective multiplex PCR amplification. In some embodiments, determining sequence read depths of amplicons is carried out by NGS.

In some embodiments, an N multiplex PCR amplification (e.g., the 1 multiplex PCR amplification) comprises a plurality of reactions, wherein for each reaction, each primer pair of the at least a subset of the N set of primer pairs is used, independently, in the reaction at one of a plurality of (such as at least 2, 3, 4, 5, 6, 7, 8, 9, 10, 11, 12, 13, 14, 15, or more) concentrations according to the N primer pair concentration profile, and wherein the plurality of reactions collectively comprise each primer pair of the at least a subset of the N set of primer pairs at each concentration of the respective plurality of concentrations. In some embodiments, the highest and lowest amplicon read depths from the 1 multiplex PCR amplification vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more). In some embodiments, the highest and lowest primer pair concentrations associated with the M primer pair concentration profile vary by at least 2-fold (such as by at least any of 3-fold, 4-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or more).

In some embodiments, the method further comprises determining sequence read depths of amplicons from the M multiplex PCR amplification (e.g., by NGS). In some embodiments, the amplicon read depths from the M multiplex PCR amplification have a CV of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amplicon read depths from the M multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less). In some embodiments, the method further comprises determining the amount of primer dimer formation (such as by NGS) following an X multiplex PCR amplification (e.g., the 1 multiplex PCR amplification) for at least a subset of primers used in the X multiplex PCR amplification, where X is any of 1 to M−1, and generating a filtered set of primer pairs based on the X set of primer pairs that excludes, for each primer dimer found to be present above a predetermined threshold, at least one of the primer pairs comprising a primer in the corresponding primer dimer. In some embodiments, the filtered set of primer pairs is used in one or more subsequent multiplex PCR amplifications. In some embodiments, the at least 50 target loci comprises at least 100 (such as at least any of 100, 200, 300, 400, 500, 600, 700, 800, 900, 1000, or more) target loci. In some embodiments, the at least 50 target loci comprises at least 500 target loci. In some embodiments, the at least 50 target loci comprises at least 1000 target loci.

### Example Assays

Once a load-balanced concentration profile has been generated using any of the methods described previously, the load balanced concentration profile may be used to produce pools of primers for use in an assay. A pool of primers includes a set of primer pairs collectively comprising at least one primer pair for each of the plurality of target loci at concentrations according to the load-balanced concentration profile. The pool of primers produced according to the load-balanced concentration profile may be used in an assay method that includes performing a multiplex PCR amplification on a nucleic acid sample. The amplicons may be sequenced (e.g., using NGS) and the resulting sequence reads used to evaluate the presence, absence, or abundance of one or more genetic markers or indicators.

In some embodiments, a method of carrying out an assay requiring the amplification of a plurality of target loci in a nucleic acid sample comprises performing a multiplex PCR amplification on the nucleic acid sample using a set of primer pairs collectively comprising at least one primer pair for each of the plurality of target loci, wherein the set of primer pairs are used in the multiplex PCR amplification at concentrations according to a load-balanced primer pair concentration profile generated according to any of the methods described herein employing carrying out M multiplex PCR amplifications. In some embodiments, the method further comprises determining sequence read depths of amplicons from the multiplex PCR amplification. In some embodiments, a read depth for each amplicon is within a threshold amount (e.g., 1%, 5%, 10%, 20%, etc.) of a corresponding target read depth. In some embodiments, the amplicon read depths from the multiplex PCR amplification have a coefficient of variance (CV) of less than 20% (such as less than any of 20%, 19%, 18%, 17%, 16%, 15%, 14%, 13%, 12%, 11%, 10%, 9%, 8%, 7%, 6%, 5%, 4%, 3%, 2%, or 1%). In some embodiments, the amplicon read depths from the multiplex PCR amplification have a 90^{th} percentile over 10^{th} percentile of less than 1.5 (such as less than any of 1.5, 1.48, 1.46, 1.44, 1.42, 1.4, 1.38, 1.36, 1.34, 1.32, 1.3, 1.28, 1.26, 1.24, 1.22, 1.2, 1.18, 1.16, 1.14, 1.12, 1.1, 1.09, 1.08, 1.07, 1.06, 1.05, 1.04, 1.03, 1.02, 1.01, or less). In some embodiments, the plurality of target loci comprises at least 50 (such as at least any of 50, 100, 200, 300, 400, 500, 600, 700, 800, 900, 1000, or more) target loci. In some embodiments, the plurality of target loci comprises at least 100 target loci. In some embodiments, the plurality of target loci comprises at least 1000 target loci.

### Nucleic Acid Sample

The nucleic acid sample used in the methods described herein can be any convenient nucleic acid sample for which it is desirable to carry out multiplex PCR amplification. In some embodiments, the nucleic acid sample is prepared from a tissue sample of a subject. In some embodiments, the nucleic acid sample is prepared from a blood sample of a subject. In some embodiments, the nucleic acid sample comprises cell-free DNA (cfDNA), such as maternal cfDNA or fetal cfDNA. In some embodiments, the nucleic acid sample comprises circulating tumor DNA (ctDNA). In some embodiments, the nucleic acid sample comprises DNA that has been subjected to bisulfite conversion, whereby unmethylated cytosines are converted to uracils. Bisulfite conversion is described in U.S. Pat. No. 8,257,950, which is incorporated herein by reference. In some embodiments, the subject is a mammal. In some embodiments, the subject is a human.

In embodiments that use cfDNA, load balancing can be particularly valuable as cfDNA is typically a limited resource. The number of cfDNA molecules in any given sample is typically low. For assays that have high target read depths for some or all loci (e.g., at least 500×, at least 1,000×, at least 10,000×, at least 20,000×, or at least 40,000×, etc.) using a load-balanced primer pool can enable efficient use of the available cfDNA molecules to provide close to the target read depth for each target loci. Conversely, if load-balanced primers are not used, the read depths for some loci will likely be significantly higher than required, effectively wasting cfDNA molecules on generating reads that are not needed for the assay to produce an accurate and reliable result. Typically, average read depths of 600× are considered high for cfDNA. The use of load-balancing can provide ultra-high average read depths (e.g., of 1000× or higher) with limited supplies of initial molecules from a sample (e.g., cfDNA from biological samples).

### EXAMPLES

Below are examples of specific embodiments for carrying out the disclosed methods. The examples are offered for illustrative purposes only and are not intended to limit the scope of the claims in any way. Efforts have been made to ensure accuracy with respect to numbers used (e.g., amounts, temperatures, etc.), but some experimental error and deviation should, of course, be allowed for.

### Example 1: Modified Primers to Reduce Primer Dimer Formation

Multiplex polymerase chain reaction (PCR) enables simultaneous amplification of multiple genomic targets in a single reaction. This can be particularly useful when there is limited volume of the input sample, splitting a sample into multiple singleplex PCRs would reduce the amount of input sample per reaction, worsening the performance of the assay. Furthermore, performing multiple reactions for a single sample can become logistically impractical in a clinical laboratory setting.

One technical challenge with performing multiplex PCR is the rate of primer dimer formation. Primer dimers are undesired amplification products that form when two primer oligonucleotides anneal to each other and amplify, instead of amplifying the desired genomic targets. In a poorly performing multiplex PCR, the products can be composed of more than 90% primer dimers. This is particularly challenging as the number of targets in the multiplex PCR increases; the number of potential primer dimers is proportional to the square of the number of primers.

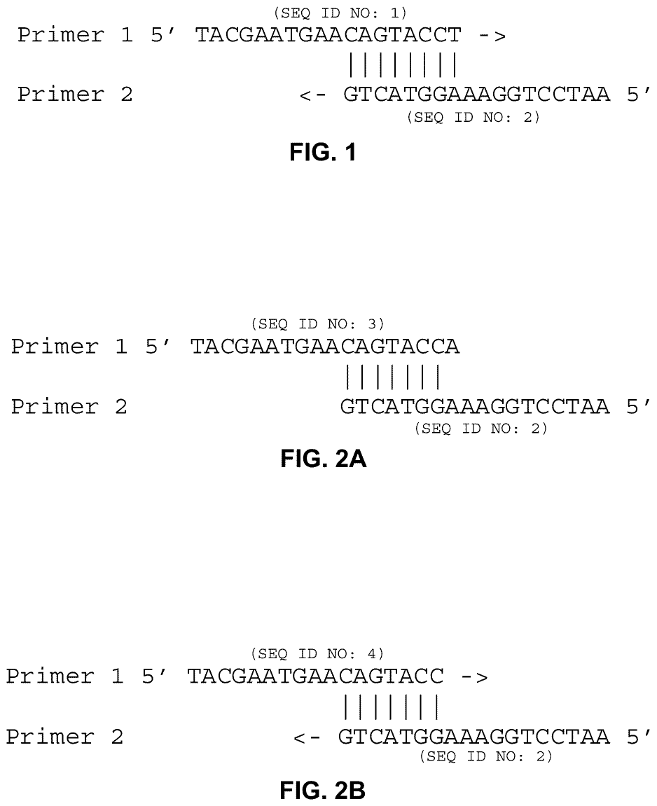

Probably the most commonly described situation is when the several bases worth of sequence at the 3′ ends of two primer oligonucleotides are perfectly complementary. During PCR, the 3′ ends anneal, and the polymerase extends to double strand both the primers (FIG. 1).

Methods to minimize this primer dimer situation include in silico detection of 3′ sequence complementarity. This can be computationally complex because one would have to check for complementarity for each combination of two primers (including one primer against itself) for a varying number of bases at the 3′ end of each primer. The complexity is a function of the number of primers squared; in silico testing of hundreds of primers is infeasible.

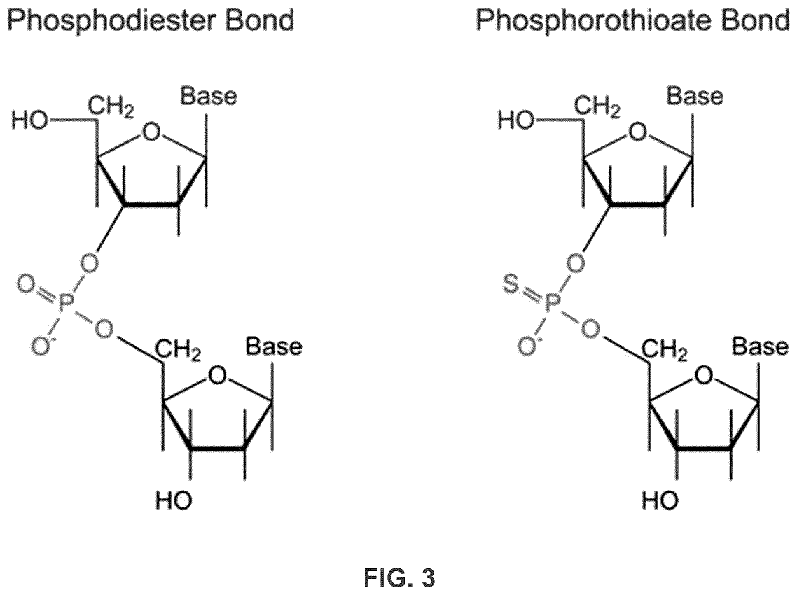

An alternative yet perhaps more common situation is when the 3′ ends of two primer oligonucleotides are not perfectly complementary. This can still lead to primer dimer formation if a polymerase with 3′ to 5′ exonuclease activity, as found in proofreading polymerases, chews back the non-complementary sequence (FIG. 2). Removal of mismatching nucleotides now results in perfectly complementary sequence on the 3′ ends as shown in FIG. 1, allowing for primer dimer amplification.

For this situation, it would be too computationally intensive to check for complementarity in silico, as the computation would now additionally require removing a varying number of bases from the 3′ ends of each primer.

We describe here methods to minimize primer dimer formation in the context of multiplex PCR, particularly for the situation described in FIG. 2.

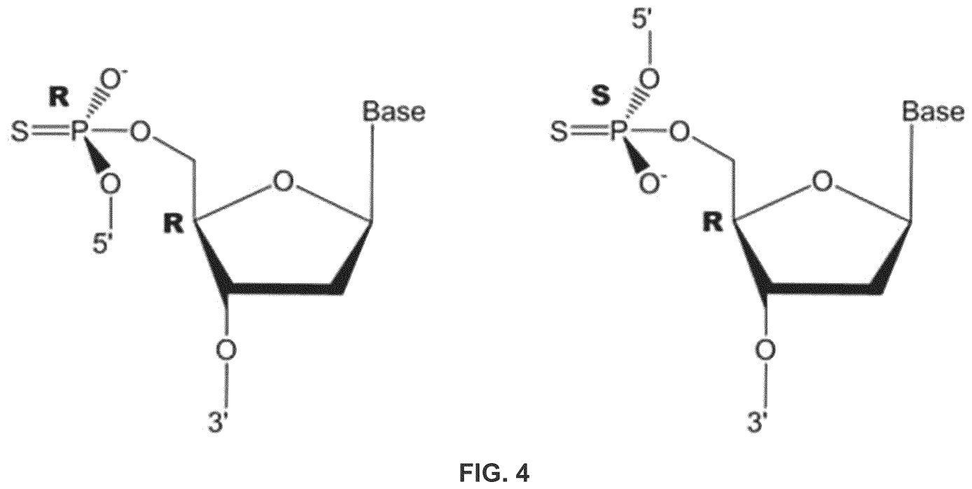

In one method, modified nucleotides can be used at the 3′ ends of primers. One example modification is to use a nucleotide with a phosphorothioate bond instead of the conventional phosphodiester bond (FIG. 3). By replacing an oxygen with a sulfur, the base becomes resistant to the polymerase's proofreading exonuclease activity.

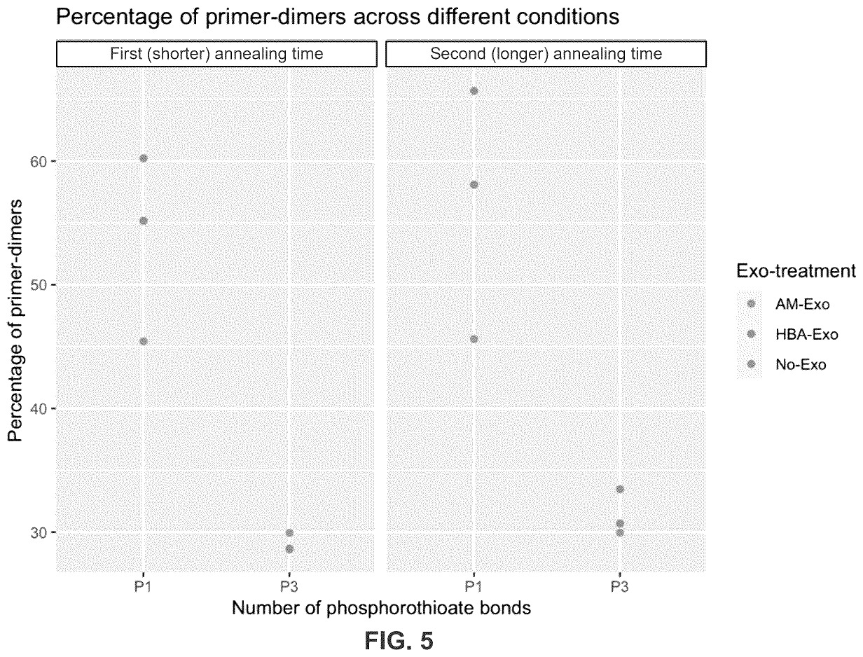

There are two possible isomers for phosphorothioate bonds, with one isomer being resistant to exonuclease activity and the other not resistant (FIG. 4). With current DNA synthesis technology, the precise phosophorothioate bond isomer that is added to the 3′ end of an oligonucleotide is random. Therefore, about 50% of synthesized primers with one nucleotide with a phosphorothioate bond on the 3′ end will not be resistant to exonuclease activity.

One solution is to add more than one nucleotide with phosphorothioate bonds to the 3′ end of each primer. This exponentially decreases the fraction of primers that have completely digestible 3′ ends. For example, with two nucleotides with phosphorothioate bonds, only ¼ of primers will be completely susceptible to 3′ exonuclease digestion. Adding more and more nucleotides with phosphorothioate bonds will thus provide marginal gains in exonuclease protection.

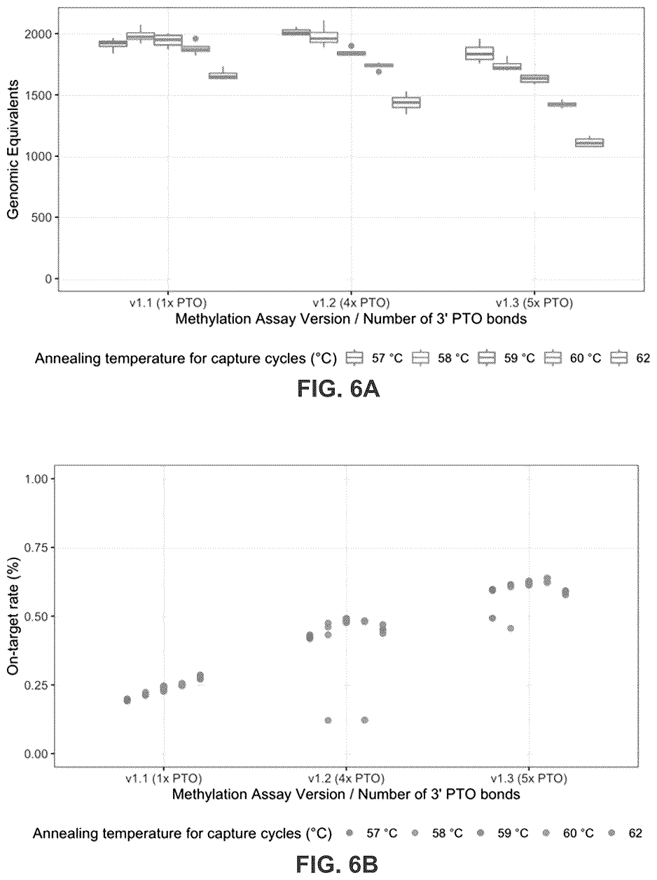

Another solution is to pre-treat primers with exonuclease to digest away primers with the non-resistant phosphorothioate bond isomer. The remaining primers can be assumed to have the resistant isomer and can be used in multiplex PCR. FIG. 5 shows the effects of different numbers of phosphorothioate bonds and pre-treatment with exonuclease on primer dimer formation. FIG. 5 shows that additional phosphorothioate bonds can reduce primer-dimer formation even without exonuclease treatment. In FIG. 5, multiplex PCR was performed using 16 primers, and primer dimer percentage was measured using next generation sequencing. FIG. 5 compares the percentage of primer-dimer formations from primers with a single phosphorothioate bond (P1) and primers with 3 phosphorothioate bonds (P3). FIG. 5 compares different exonuclease treatment protocols (AM-Exo and HBA-Exo) with no exonuclease treatment at all (No-Exo) FIG. 5 shows that the percentage of primer-dimer formation is significantly lower in the P3 group even without exonuclease treatment. FIG. 5 also illustrates that there is generally a greater amount of primer-dimer formation when a longer annealing time is used, but that the inclusion of additional phosphorothioate bonds and exonuclease pre-treatment both reduce primer-dimer formation regardless of annealing time.

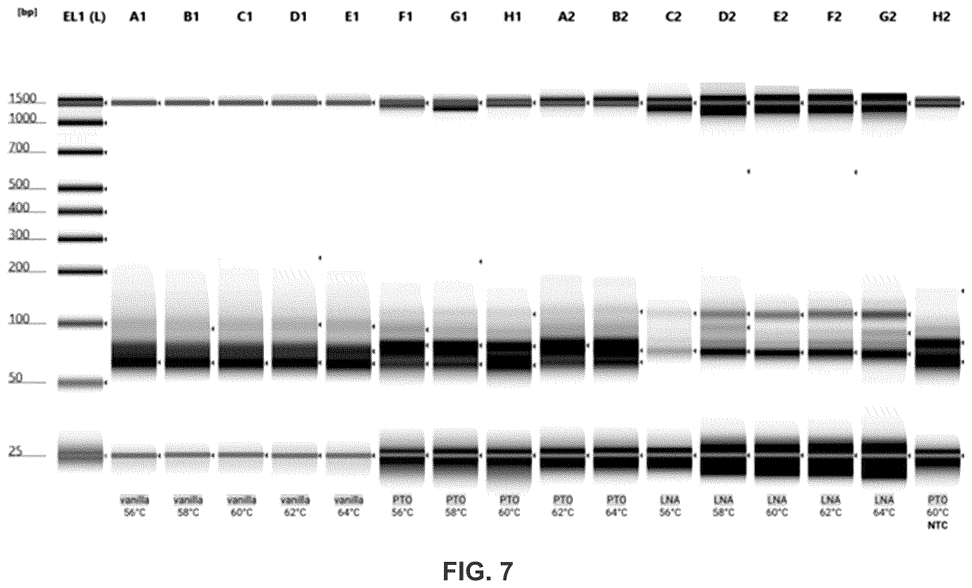

Multiplex PCR reactions with up to five consecutive phosphorothioate bonds at the 3′ end of each primer were tested. FIGS. 6A and 6B illustrate that there are consistent improvements in the on-target rate for a mix of more than 500 primer pairs as the number of phosphorothioate bonds increases, indicating that primer dimer formation continues to decrease despite protecting exponentially fewer primers with each additional phosphorothioate bond. Notably, while adding phosphorothioate bonds decreases the melting temperature of primers which could necessitate reducing the annealing temperature of the PCR to maintain comparable yield of the assay, the reaction results in high on-target rate despite lower annealing temperatures favoring primer dimer formation in general.

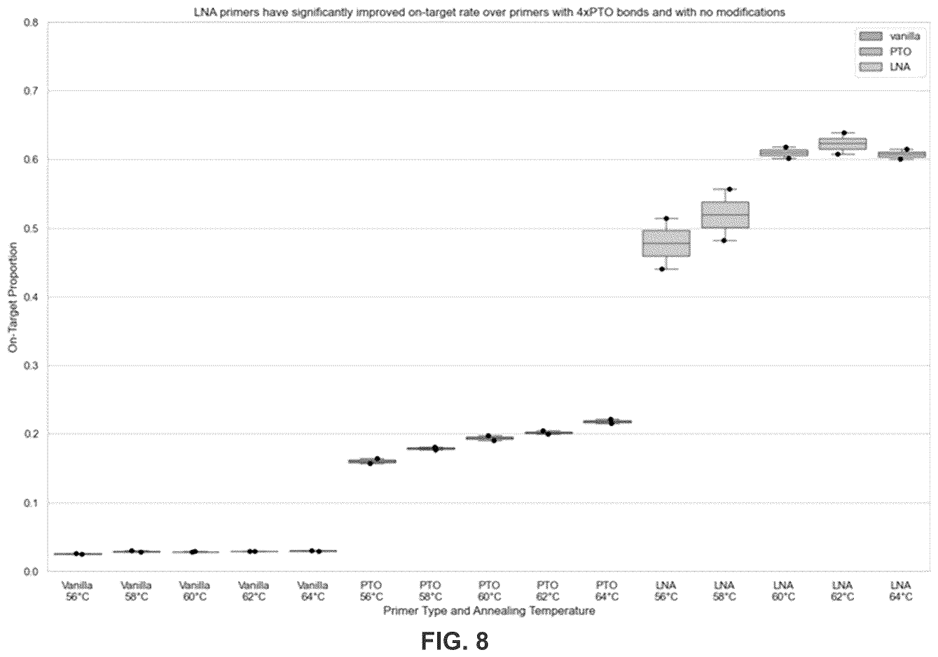

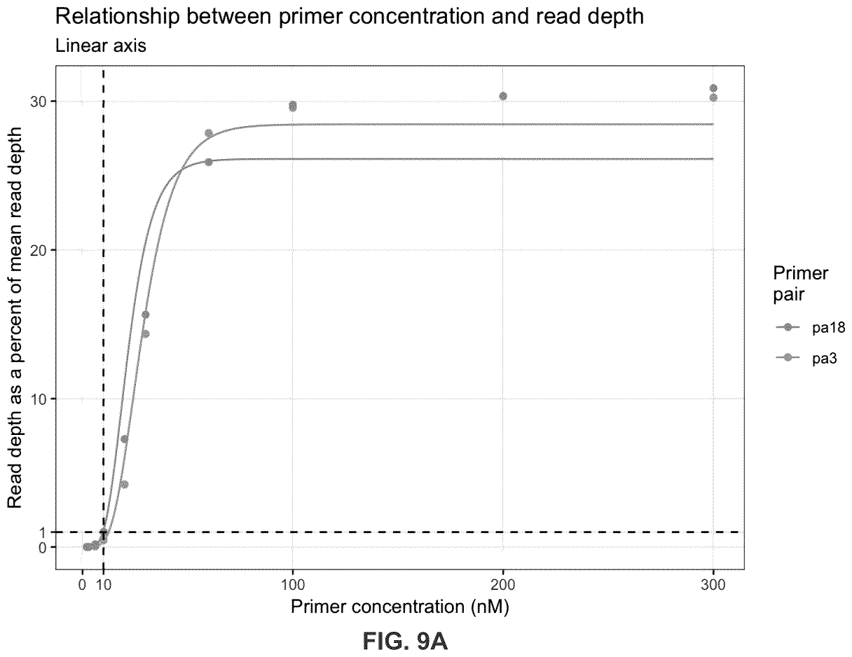

LNAs can be used in place of unmodified nucleotides at the 3′ end of primers to resist exonuclease activity. It has been shown that placing a locked nucleic acid at the penultimate 3′ base greatly increases 3′ exonuclease resistance. Multiplex PCR using primers containing locked nucleic acids was tested. FIGS. 7 and 8 illustrate the testing of a 16 primer pair multiplex PCR and demonstrate improved on-target rate for primers with LNAs compared to primers without any modifications, as measured by gel electrophoresis and next-generation sequencing.

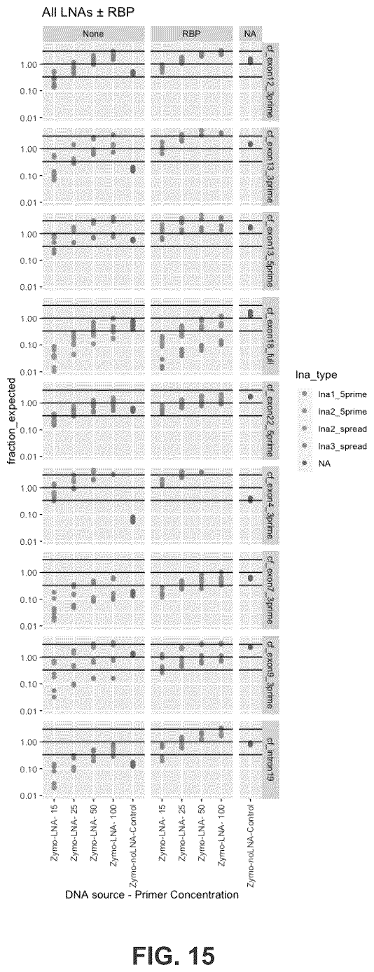

In some embodiments, LNAs can be used to balance amplification. In some embodiments, LNAs can be used to improve amplification efficiency or amplification evenness. FIG. 15 shows a set of poorly performing amplicons in which the primers have been modified to include LNAs in 4 different designs, including (1) a single LNA approximately 5 bases from the 5′ end of the core binding region (lna1_5prime), (2) the same as (1) but with an additional LNA 2-3 bases 5′ of the initial one (lna2_5prime), (3) the same as (1) but with an additional LNA 5-7 bases from the 3′ end (lna2_spread), and (4) LNAs combining the locations from (1-3) resulting in a total of 3 LNAs in the oligonucleotide (lna3_spread). These oligonucleotides were added at varying concentrations to multiplex reactions and the reactions were performed on bead extracted cfDNA, bead extracted cfDNA with an RBP step to further purify contaminants, or on cell-line based highly pure shDNA as a control. A 100 nM non-LNA primer was also included as a control for baseline performance. FIG. 15 shows that LNAs in primers can provide equal or greater amplification efficiency comparing to original primer designs and restore amplification efficiency and balance in under-performing amplicons with poor response curves.

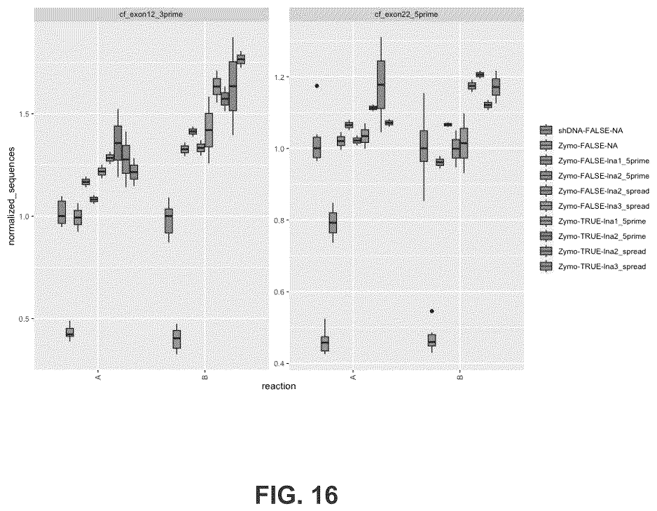

In some embodiments, LNAs can be used to improve capture efficiency or capture performance. FIG. 16 illustrates two amplicons which were examined with or without RBP and containing different LNA primer compositions across 2 different reactions. All conditions for each amplicon were normalized the shDNA (red bar) and variation represents technical replicates. The orange bar represents samples that underwent cfDNA extraction from plasma and used non-LNA primers showing greater than 50% molecular capture loss. For all designs, the number of molecules captured by LNA primers was equal or greater than the control shDNA condition with only slight enhancement by further purification (Zymo-RBP TRUE conditions). FIG. 16 shows that LNAs can improve capture efficiency and restore full capture to amplicons that were underperforming due to the post-purification conditions.

### Example 2: Effect of Primer Concentration on Read Depth

This Example looks at how changing primer concentration effects read depth in a multiplex PCR reaction. Two datasets were generated to look into this phenomenon. The first dataset looks at a broad range of primer concentrations for two primer pairs. The second dataset looks at five primer concentrations for all 20 primer pairs. All changes in primer concentration were done in a background of 10 nM of 19 other primer pairs. The general reaction conditions are as follows: 50 ng NA12878 gDNA; 10 nM Primers: 20 primer pairs (one excluded from analysis); Enrichment and amplification PCR: 1. 98 C-5 min, 2. 20 times: 98 C-10 s, 60 C-2 min, 72 C-30 s, 3. 72 C-1 min, 4. 4 C-Hold; Indexing PCR: 1. 98 C-60 s, 2. 2 times: 98 C-15 s, 54 C-20 s, 72 C-20 s, 3. 4 times: 98 C-15 s, 70 C-20 s, 72 C-20 s, 4. 72 C-1 min, 5. 4 C-Hold.

To get an understanding of the relationship between primer pair concentration and read depth, two primer pairs were selected and their concentrations were varied as follows: 0, 1, 2, 3, 6, 10, 20, 30, 60, 100, 200, and 300 nM. One of the primer pairs (pa18) had a read depth equivalent to the average read depth of all primer pairs. The other primer pair (pa3) had a read depth much lower than the average read depth. The 19 other primer pairs were held constant at 10 nM.

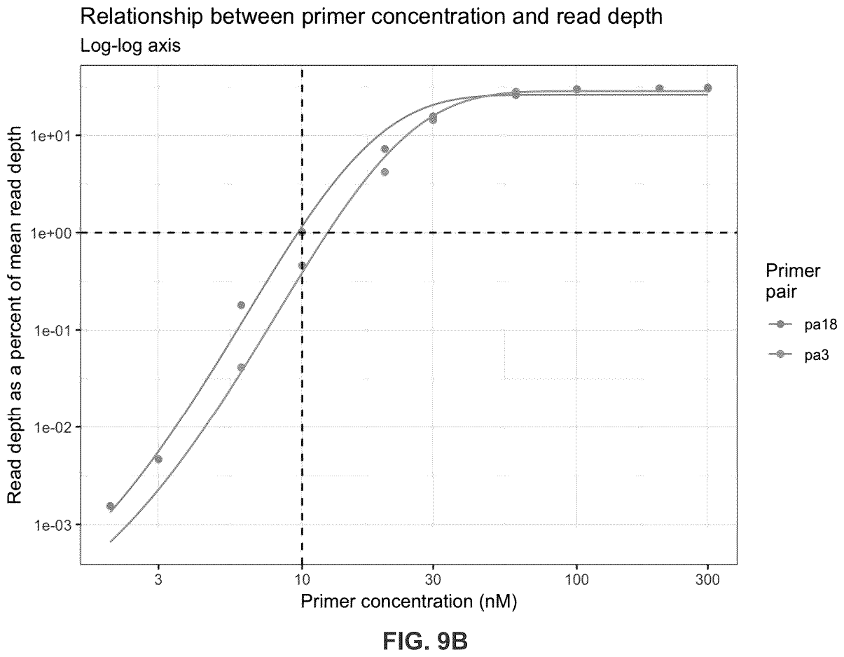

The first graph (FIG. 9A) was plotted using linear axes to give a sense of the shape of these curves. The y-axis is the read depth of the primer pair being varied compared to the mean read depth of the 18 other primer pairs that are present at 10 nM in the reaction. pa18, which was selected to have average read depth, had a value of 1 when used at 10 nM.

It is more informative to look at the log-log presentation of the data in the second graph (FIG. 9B). ˜4 logs of expression are covered with pa18. Both pa18 and pa3 are missing lower concentration data points due to insufficient read depth. A 5 reads threshold is present in this data. This suggests that an even larger range of concentrations can be measured with higher read depth. However, it also means that there is significant read noise in the last data point of each data set. We also see that the difference in amplification efficiency between pa18 and pa3 disappears at higher primer concentrations.

To fit the data, a model of how final read depth relates to the primer concentration was created. PCR reaction can be modelled as follows: final=initial(1+eff ([P]))ⁿ, where n is the number of PCR cycles and eff([P]) is the replication efficiency of the PCR reaction and is dependent on primer concentration.

To determine how the PCR efficiency is related to primer concentration, a single cycle of a PCR reaction was modelled. It was assumed that a PCR reaction could be modelled as a bimolecular reaction between single-stranded DNA and primer:

$$\frac{d [ ds ]}{dt} = {k}_{cat} [ ss ] [ P ] ,$$

where [ds] is the concentration of double-stranded DNA, [ss] is the concentration of single-stranded DNA, [P] is the primer concentration, and k_{cat} is the rate constant of the reaction. Solving for [ds] gives [ds]=[ss]₀(1−e^{-k}^{_{cat}}^{t[P]}). This enables calculation of

$$eff ( [ P ] ) = \frac{{[ ds ]}_{f}}{{[ ss ]}_{0}} = 1 - {e}^{- {k}_{cat} {t}_{f} [ P ]} .$$

Substituting eff([P]) into the PCR model above gives final=initial(2−e^{-k}^{_{cat}}^{t}^{_{f}}^{[P]})ⁿ. The model derived above can be simplified to the following form: reads=b(2−a^{-[P]})²⁰. The fits in this Example employed this model and a nonlinear least-squares fit. Fits were performed in the log-log space to weight low and high concentrations equally.

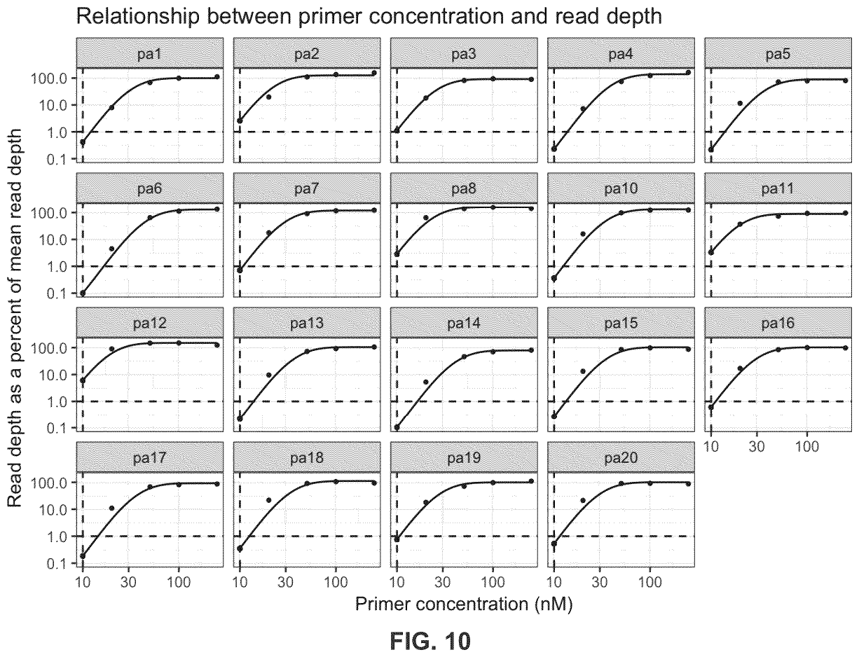

We next performed a similar experiment for all 20 of the primer pairs in this assay. Primers were used at the following concentrations (nM): 10, 20, 50, 100, 250. These reactions were conducted in a background of 10 nM of all other primer pairs. Data were plotted and fit as above on a log-log axis (FIG. 10).

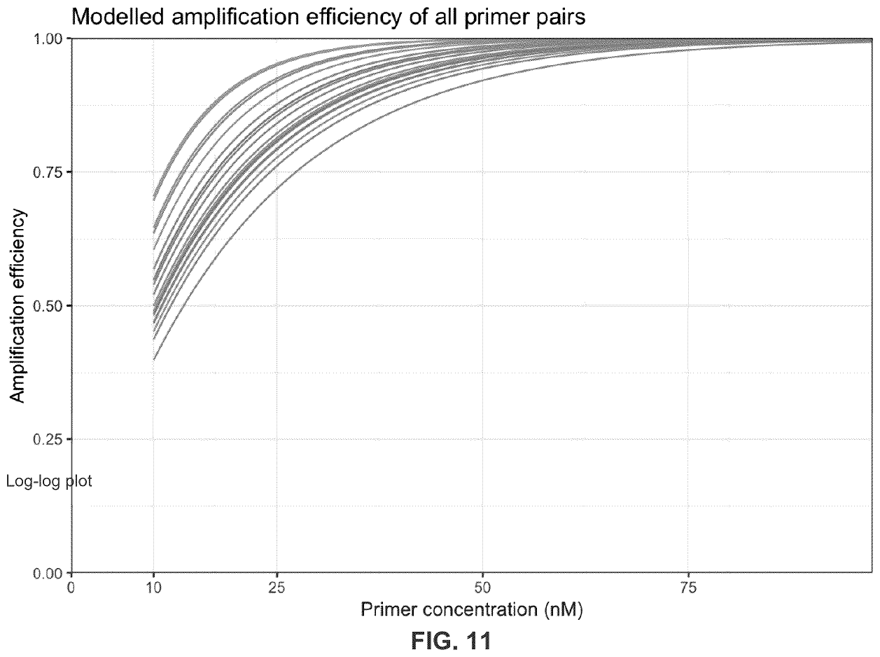

The amplification efficiency can be derived from each of the fits in FIG. 10. The amplification efficiencies for each primer pair as a function of concentration are shown in FIG. 11. It is clear from FIG. 11 that a 10 nM baseline concentration provides a poor amplification efficiency. This can have several negative results. Amplification efficiency is related to capture efficiency. Therefore, using a concentration of 10 nM is likely t result in capture of only about half of the molecules in the reaction. There is also likely to be higher bias due to sequence differences in the amplified template at this amplification efficiency. This is because at lower amplification efficiencies the reaction is likely more sensitive to perturbations, including differences in sequence identity. However, this may be mitigated with normalization to a control for bias due to sequence difference between a spike-in and a template, so these relatively low concentrations are still appropriate for some primer pairs in some assays.

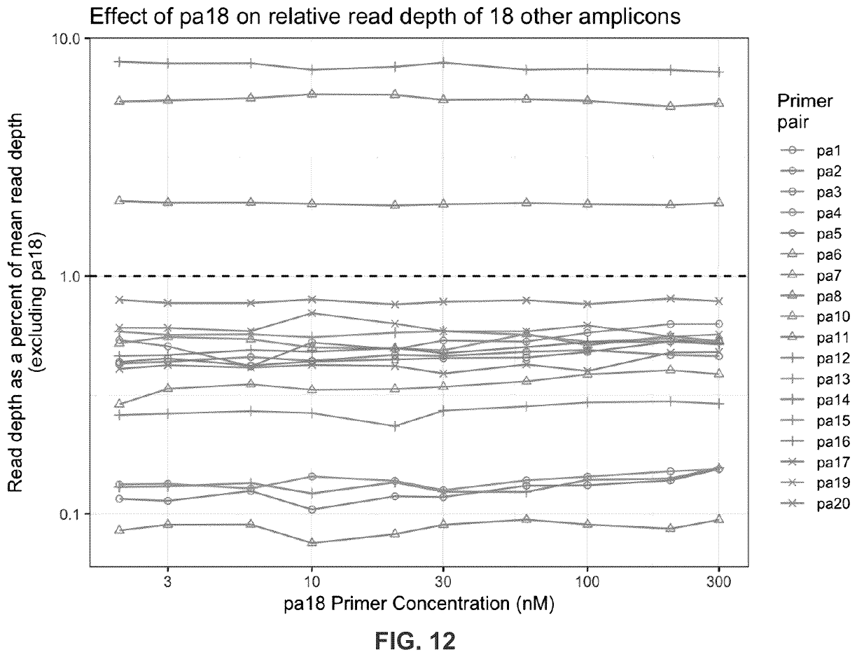

FIG. 12 illustrates that the concentration of one primer pair can be changed without causing significant second order effects on the behavior of other primer pairs. Specifically, FIG. 12 shows the mean read depth for all of the primer pairs in the test set of 20 primers (except pa18) with a constant concentration as the concentration of pa18 is varied. It can be seen the mean read depths of all 19 other primer pairs remained substantially constant with changes to the concentration of pa18. This was a surprising result as multiplex PCR often exhibits significant second order effects between primer pairs.

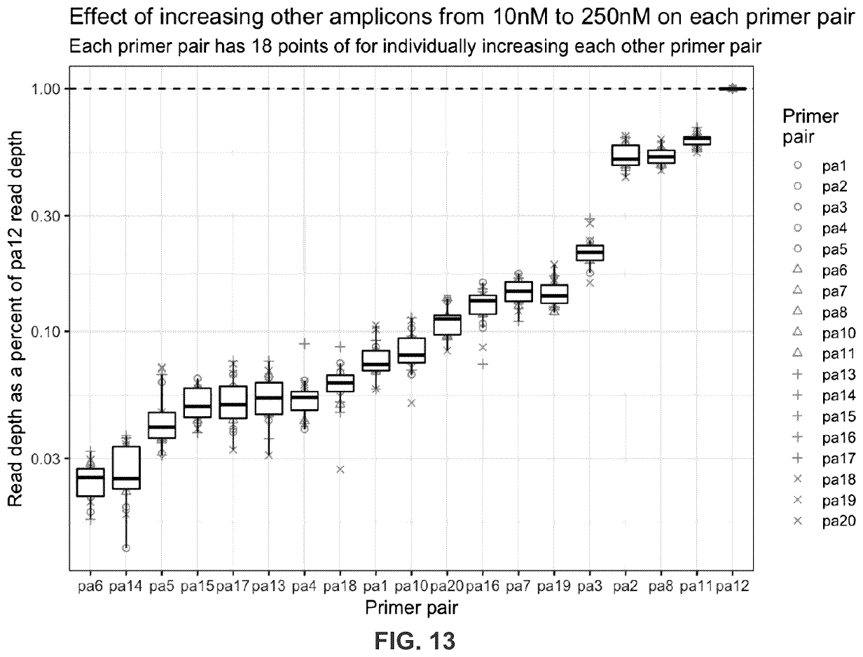

FIG. 13 illustrates a more comprehensive data set of the secondary interactions between primer pairs in the test set of 20 primer pairs. All 20 primers were added at 250 nM in 20 different reactions. FIG. 13 is a plot of each primer pair's its read depth when each of the other 19 primer pairs were added at 250 nM. The read depth is plotted as a percent of pa12 reads. This results in a measurement of secondary effects of 19 other primer pairs. It can be seen from the plot that there are not any extreme outliers in the data, suggesting that there are not any significant secondary interactions between these primers when one is at 10 nM and the other is 250 nM.

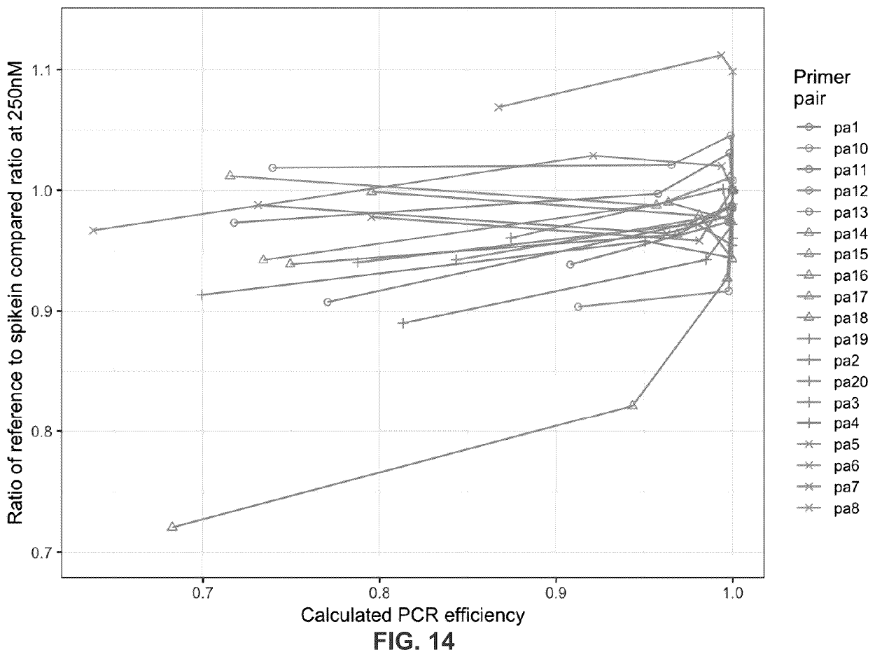

FIG. 14 illustrates the impact of amplification efficiency on amplification bias due to sequence identity. FIG. 14 plots the reference to spike-in ratio normalized for that ratio at the maximum primer against amplification efficiency for each primer concentration (as calculated previously based on the previous fits described with reference to FIGS. 10 and 11). If there was no relationship between amplification efficiency and amplification bias, the plots in FIG. 14 would be straight, vertical lines. It can be seen that while there is some impact on bias from changes in amplification efficiency, there is not a consistent pattern between primer pairs and the impact is relatively small for most primer pairs.

### ADDITIONAL CONSIDERATIONS

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, molecular biology and nucleic acid chemistry and hybridization described herein are those well-known and commonly used in the art.

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the elements or components are present unless it is obvious that it is meant otherwise.

Where values are described as “approximate” or “substantially” (or their derivatives), such values should be construed as accurate +/−10% unless another meaning is apparent from the context From example, “approximately ten” should be understood to mean “in a range from nine to eleven.”

The terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Whereas various specific embodiments have been illustrated and described, the above specification is not restrictive. Many variations will become apparent to those skilled in the art upon review of this specification. It will be appreciated that various changes can be made without departing from the spirit and scope of the disclosure. As such, the scope of protection should be limited only by the following claims.

1. A method of amplifying at least 100 target loci in a nucleic acid sample, comprising performing multiplex PCR amplification on the nucleic acid sample using a pool of primers comprising a first set of primers comprising at least one primer pair for each target locus of the at least 100 target loci, wherein at least one primer in the first set of primers in the pool of primers comprises at least one exonuclease-resistant internucleotide bond, wherein the pool of primers was
(i) treated with an enzyme having a 3′ to 5′ exonuclease activity prior to performing the multiplex PCR amplification, thereby removing at least 50% of primers that do not have at least one exonuclease-resistant nucleotide bond, and (ii) treated to inactivate the enzyme having 3′ to 5′ exonuclease activity,
wherein the primers remaining in the pool of primers after treatment with the enzyme having a 3′ to 5′ exonuclease activity amplify a set of amplicons.

2. The method of claim 1, wherein each of the at least one primer in the first set of primers comprises an exonuclease-resistant internucleotide bond between:
(a) a nucleotide at the 3′-terminal position (0 position) and the nucleotide immediately upstream of the 0 position (−1 position);
(b) a nucleotide at the −1 position and the nucleotide immediately upstream of the −1 position (−2 position);
(c) a nucleotide at the −2 position and the nucleotide immediately upstream of the −2 position (−3 position); or
(d) a nucleotide at the −3 position and the nucleotide immediately upstream of the −3 position (−4 position).

3. (canceled)

4. The method of claim 1, wherein following enzymatic treatment less than 1% of the primers remaining in the pool of primers do not comprise at least one exonuclease-resistant internucleotide bond.

5. The method of claim 1, wherein the exonuclease-resistant internucleotide bond is a phosphorothioate bond.

6. The method of claim 1, wherein at least 90% of the primers in the pool of primers comprises an exonuclease-resistant internucleotide bond.

7. The method of claim 1, wherein the amount of primer dimer formation in the multiplex PCR amplification is less than 70% of the amount of primer dimer formation that would occur in a corresponding multiplex PCR amplification where each of the primers in the pool of primers has only phosphodiester internucleotide bonds.

8. The method of claim 1, wherein the percent of primer dimers in the products of the multiplex PCR amplification is less than 50%.

9. (canceled)

10. The method of claim 1, wherein the pool of primers further comprises a second set of primers and the second set of primers comprises at least one primer having four or more phosphorothioate bonds, each phosphorothioate bond being an Rp isomer or an Sp isomer, such that statistically a predetermined percentage of the second set of primers of the pool of primers have at least one Sp isomer phosphorothioate bond, wherein the second set of primers having at least one exonuclease-resistant internucleotide bond includes those of the second set of primers that have at least one Sp isomer phosphorothioate bond.

11. The method of claim 1, wherein at least some of the first set of primers in the pool of primers comprise an exonuclease-resistant nucleotide.

12. The method of claim 11, wherein, for a primer that has an exonuclease-resistant nucleotide, the exonuclease-resistant nucleotide is at:
(a) the 3′-terminal position (0 position);
(b) the nucleotide immediately upstream of the 0 position (−1 position);
(c) the nucleotide immediately upstream of the −1 position (−2 position); or
(d) the nucleotide immediately upstream of the −2 position (−3 position).

13. The method of claim 11, wherein the exonuclease-resistant nucleotide is a nucleotide having a sugar moiety with a bridge connecting the 2′ oxygen and 4′ carbon.

14. The method of claim 1, wherein the pool of primers is a load-balanced set of primer pairs, wherein concentrations of the primer pairs in the first set of primers was determined by a process comprising:
(i) performing a multiplex PCR amplification on a first balancing nucleic acid sample using an initial pool of primers comprising a set of initial primer pairs collectively comprising at least one primer pair for each of the plurality of target loci, wherein each initial primer pair is present in the initial pool of primers at a concentration according to a first primer pair concentration profile, thereby generating amplicons corresponding to the different primer pairs;
(ii) determining sequence read depths of amplicons from the multiplex PCR amplification corresponding to the different primer pairs to generate a first amplification profile;
(iii) generating an updated primer pair concentration profile based on the first amplification profile, wherein concentrations in the updated primer pair concentration profile are selected based on the first amplification profile to adjust the sequence read depths to be closer to corresponding target sequence read depths for the primer pairs; and
(iv) determining the concentrations of the primer pairs for the load-balanced set of primer pairs based on the updated primer pair concentration profile.

15. A method of amplifying a plurality of target loci in a nucleic acid sample, comprising:
performing multiplex PCR amplification on the nucleic acid sample using a pool of primers comprising a first set of primers collectively comprising at least one primer pair for each target locus of the plurality of target loci, wherein the first set of primers comprises a plurality of primers each having at least one exonuclease-resistant nucleotide, wherein:
(i) the plurality of target loci comprises at least 100 target loci; or
(ii) the at least one exonuclease-resistant nucleotide comprises a plurality of exonuclease-resistant nucleotides,
and wherein the pool of primers was (i) treated with an enzyme having a 3′ to 5′ exonuclease activity prior to performing the multiplex PCR amplification, thereby removing at least 50% of primers that do not have at least one exonuclease-resistant nucleotide bond, and (ii) treated to inactivate the enzyme having 3′ to 5′ exonuclease activity,
wherein the primers remaining in the pool of primers after treatment with the enzyme having a 3′ to 5′ exonuclease activity amplify a set of amplicons.

16. The method of claim 15, wherein the plurality of primers in the first set of primers each comprises an exonuclease-resistant nucleotide at:
(a) the 3′-terminal position (0 position);
(b) the nucleotide immediately upstream of the 0 position (−1 position);
(c) the nucleotide immediately upstream of the −1 position (−2 position); or
(d) the nucleotide immediately upstream of the −2 position (−3 position).

17. (canceled)

18. The method of claim 15, wherein following enzymatic treatment less than 1% of the primers remaining in the pool of primers do not comprise at least one exonuclease-resistant nucleotide.

19. The method of claim 15, wherein the exonuclease-resistant nucleotide is a nucleotide having a sugar moiety with a bridge connecting the 2′ oxygen and 4′ carbon.

20. The method of claim 15, wherein the plurality of primers comprises at least 90% of the primers in the pool of primers.

21. The method of claim 15, wherein the amount of primer dimer formation in the multiplex PCR amplification is less than 70% of the amount of primer dimer formation in a corresponding multiplex PCR amplification where each of the primers in the pool of primers has only natural nucleotides.

22. The method of claim 15, wherein the percent of primer dimers in the products of the multiplex PCR amplification is less than 50%.

23. (canceled)

24. The method of claim 15, wherein the pool of primers further comprises a second set of primers, and at least some of the second set of primers in the pool of primers have an exonuclease-resistant internucleotide bond.

25. The method of claim 24, wherein, for a primer that has an exonuclease-resistant internucleotide bond, the exonuclease-resistant bond is between:
(a) a nucleotide at the 3′-terminal position (0 position) and the nucleotide immediately upstream of the 0 position (−1 position);
(b) a nucleotide at the −1 position and the nucleotide immediately upstream of the −1 position (−2 position);
(c) a nucleotide at the −2 position and the nucleotide immediately upstream of the −2 position (−3 position); or
(d) a nucleotide at the −3 position and the nucleotide immediately upstream of the −3 position (−4 position).

26. The method of claim 24, wherein the exonuclease-resistant internucleotide bond is a phosphorothioate bond.

27. The method of claim 24, wherein each of the second set of primers has four or more phosphorothioate bonds, each phosphorothioate bond being an Rp isomer or an Sp isomer, such that statistically a predetermined percentage of the second set of primers of the pool of primers have at least one Sp isomer phosphorothioate bond, wherein the second set of primers having at least one exonuclease-resistant internucleotide bond includes those of the second set of primers that have at least one Sp isomer phosphorothioate bond.

28. A method of amplifying a plurality of target loci in a nucleic acid sample, comprising performing multiplex PCR amplification on the nucleic acid sample using a pool of primers comprising a set of primers collectively comprising at least one primer pair for each locus of the plurality of target loci,
wherein each of a plurality of primers in the set of primers has at least one exonuclease-resistant nucleotide,
wherein:
(i) the plurality of target loci comprises at least 50 target loci; or
(ii) the at least one exonuclease-resistant nucleotide comprises a plurality of exonuclease-resistant nucleotides,
wherein the pool of primers is a load-balanced set of primer pairs, wherein concentrations of the primer pairs was determined by a process comprising:
(i) performing a multiplex PCR amplification on a first balancing nucleic acid sample using an initial pool of primers comprising a set of initial primer pairs collectively comprising at least one primer pair for each of the plurality of target loci, wherein each initial primer pair is present in the initial pool of primers at a concentration according to a first primer pair concentration profile, thereby generating amplicons corresponding to the different primer pairs;
(ii) determining sequence read depths of amplicons from the multiplex PCR amplification corresponding to the different primer pairs to generate a first amplification profile;
(iii) generating an updated primer pair concentration profile based on the first amplification profile, wherein concentrations in the updated primer pair concentration profile are selected based on the first amplification profile to adjust the sequence read depths to be closer to corresponding target sequence read depths for the primer pairs; and
(iv) determining the concentrations of the primer pairs for the load-balanced set of primer pairs based on the updated primer pair concentration profile.

29. The method of claim 15, wherein at least some of the primers include at least one exonuclease-resistant nucleotide and at least one exonuclease-resistant internucleotide bond, thereby balancing impacts on primer annealing temperature and sequence read depths of amplicons caused by the at least one exonuclease-resistant nucleotide and the at least one exonuclease-resistant internucleotide bond.

30. The method of claim 29, wherein the at least one exonuclease-resistant nucleotide is a nucleotide having a sugar moiety with a bridge connecting the 2′ oxygen and 4′ carbon and the at least one exonuclease-resistant internucleotide bond is a phosphorothioate bond.


SEQUENCE LISTING (4 sequences)

SEQ ID NO: 1 - DNA, 18 bp, Unidentified
  source 1..18: mol_type = genomic DNA; note = synthetic construct
tacgaatgaacagtacct

SEQ ID NO: 2 - DNA, 18 bp, Unidentified
  source 1..18: mol_type = other DNA; note = synthetic construct
gtcatggaaaggtcctaa

SEQ ID NO: 3 - DNA, 18 bp, Unidentified
  source 1..18: mol_type = other DNA; note = synthetic construct
tacgaatgaacagtacca

SEQ ID NO: 4 - DNA, 17 bp, Unidentified
  source 1..17: mol_type = other DNA; note = synthetic construct
tacgaatgaacagtacc